(19) United States
(12) Patent Application Publication
(10) Pub. No.: US 2026/0240669 A1
(43) Pub. Date: Aug. 20, 2026

(54) THERAPEUTIC BIO-ABSORBABLE POLYMERIC SCAFFOLD WITH DRUG-LOADED MICRO-NEEDLES FOR DRUG DELIVERY

(71) Applicant: BECTON, DICKINSON AND COMPANY, Franklin Lakes, NJ (US)

(72) Inventors: Aaron Blank, Chandler, AZ (US); Hiep Quang Do, Chandler, AZ (US); Chad C. Van Liere, Phoenix, AZ (US); Sarfaraz Shamji, San Antonio, TX (US)

(73) Assignee: BECTON, DICKINSON AND COMPANY, Franklin Lakes, NJ (US)

(21) Appl. No.: 19/162,028

(22) PCT Filed: Mar. 10, 2023
(86) PCT No.: PCT/US23/64102

§ 371 Date: Sep. 3, 2025

(51) Int. Cl. A61F 2/915; A61F 2/848; A61F 2/958

(52) U.S. Cl. CPC A61F 2/915; A61F 2/848; A61F 2/958; A61F 2002/8483; A61F 2250/0067

(57) ABSTRACT

The present disclosure concerns microneedles around the exterior surface of a radially expandable medical device that puncture or pierce the vessel wall as the device expands. The microneedles can be embedded and/or coated with one or more therapeutic agents in order to provide localized delivery to the tissue where the medical device is deployed. In further aspects, the microneedles can be of a metal or a biopolymer that is degraded and/or absorbed within a subject. In some aspects, the microneedles may project from an underlying balloon. A protective covering is also contemplated for maneuvering the device within a subject.

### TECHNICAL FIELD

The present specification generally relates medical devices that deliver drug-loaded micro-needles into the tunica media layer of a vessel wall for localized drug delivery to prevent restenosis.

### BACKGROUND

Vascular medical devices are deployed into the lumen of a vessel to provide structural support. Some, like balloon catheters, are temporary, while others such as stents, are permanent or semi-permanent. That a vascular device is deployed in an area requiring treatment makes the incorporation of therapeutics attractive for localized delivery. Drug-eluting devices typically therefore include a coating designed to release therapeutics to the vessel walls at and around the site of deployment. However, the diseased or distressed state of the tissue and the volatile flow of fluid within the lumen can render success challenging. Thus a need for improved delivery mechanisms is needed.

### SUMMARY

A 1^{st} aspect of the present disclosure, either alone or in combination with any other aspect herein, concerns a medical device comprising a radially-expandable scaffold structure with at least one microneedle positioned circumferentially thereon, wherein the microneedle comprises a coating layer on at least a portion of an outer surface, wherein the coating layer comprises at least one therapeutic agent.

A 2^{nd} aspect of the present disclosure, either alone or in combination with any other aspect herein, concerns the medical device of the 1^{st} aspect, wherein the radially-expandable scaffold structure comprises a stent.

A 3^{rd} aspect of the present disclosure, either alone or in combination with any other aspect herein, concerns the medical device of the 1^{st} or 2^{nd} aspects, wherein radially-expandable scaffold structure is adhered to an underlying balloon.

A 4^{th} aspect of the present disclosure, either alone or in combination with any other aspect herein, concerns the medical device of the 1^{st} or 2^{nd} aspects, wherein the at least one microneedle is attached to the radially-expandable scaffold structure at a hinge or joint structure thereof.

A 5^{th} aspect of the present disclosure, either alone or in combination with any other aspect herein, concerns the medical device of the 1^{st} or 2^{nd} aspects, wherein the at least one microneedle is attached to the radially-expandable scaffold structure along an arm thereof.

A 6^{th} aspect of the present disclosure, either alone or in combination with any other aspect herein, concerns the medical device of the 1^{st}, 2^{nd}, 4^{th}, or 5^{th} aspects, wherein the at least one microneedle is adhered or welded to the radially-expandable scaffold structure.

A 7^{th} aspect of the present disclosure, either alone or in combination with any other aspect herein, concerns the medical device of the 1^{st}, 2^{nd}, 4^{th}, or 5^{th} aspects, wherein the microneedle is part of the radially-expandable scaffold structure.

An 8^{th} aspect of the present disclosure, either alone or in combination with any other aspect herein, concerns the medical device of the 7^{th} aspect, wherein there is no point of connection between the radially-expandable scaffold structure and the at least one microneedle.

A 9^{th} aspect of the present disclosure, either alone or in combination with any other aspect herein, concerns the medical device of the 1^{st} or 2^{nd} aspects, wherein the at least one microneedle extends through the radially-expandable scaffold structure from an external surface of an underlying balloon.

A 10^{th} aspect of the present disclosure, either alone or in combination with any other aspect herein, concerns the medical device of any preceding aspects, wherein the at least one microneedle is of the same material as the radially-expandable scaffold.

An 11^{th} aspect of the present disclosure, either alone or in combination with any other aspect herein, concerns the medical device of any prior aspect, wherein the at least one microneedle is of biopolymer material, wherein the biopolymer material is selected from the group consisting of poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, modified polyethylene glycol, or a copolymer or mixture thereof and wherein optionally the therapeutic agent is embedded therein.

A 12^{th} aspect of the present disclosure, either alone or in combination with any other aspect herein, concerns the medical device of any of the 1^{st} to 11^{th} aspects, wherein the microneedle is of a metal selected from stainless steel, titanium, nickel, tantalum, platinum, palladium, or nitinol.

A 13^{th} aspect of the present disclosure, either alone or in combination with any other aspect herein, concerns the medical device of the 1^{st} or 2^{nd} aspects, wherein the at least one microneedle is connected to an outer surface of the radially-expandable scaffold structure through a detachable junction.

A 14^{th} aspect of the present disclosure, either alone or in combination with any other aspect herein, concerns the medical device of the 1^{st} or 2^{nd} aspects, further comprising a hydrophilic-coated thermoplastic layer that covers the coating layer, the radially-expandable scaffold structure, and the at least one microneedle.

A 15^{th} aspect of the present disclosure, either alone or in combination with any other aspect herein, concerns the medical device of the 14^{th} aspect, wherein the hydrophilic-coated thermoplastic layer comprises a biopolymer material selected from the group consisting of poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, modified polyethylene glycol, or a copolymer or mixture thereof.

A 16^{th} aspect of the present disclosure, either alone or in combination with any other aspect herein, concerns the medical device of the 1^{st} or 2^{nd} aspects, wherein the coating layer further comprises nanoparticles with the at least one therapeutic agent loaded therein.

A 17^{th} aspect of the present disclosure, either alone or in combination with any other aspect herein, concerns the medical device of the 16^{th} aspect, wherein the nanoparticles comprise a biopolymer selected from poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, modified polyethylene glycol, or a copolymer or mixture thereof.

An 18^{th} aspect of the present disclosure, either alone or in combination with any other aspect herein, concerns the medical device of any of the 1^{st} to 17^{th} aspects, wherein the therapeutic agent is paclitaxel, rapamycin, daunorubicin, 5-fluorouracil, doxorubicin, bosutinib, ceritinib, crizotinib, gefitinib, ruxolitinib, imatinib, axitinib, nilotinib, trametinib, afatinib, ibrutinib, cabozantinib, imatinib, lenvatinib, sunitinib, regorafenib, sorafenib, vandetanib, dasatinib, pazopanib, Y27632, CA3, verteporfin, VGLL4 peptide, nintedanib, avapritinib, abemaciclib, erdafitinib, fedratinib, palbociclib, pemigatinib, irinotecan, bevasizumab, cetuxamab, biolimus (biolimus A9), everolimus, zotarolimus, tacrolimus, dexamethasone, prednisolone, corticosterone, cisplatin, vinblastine, lidocaine, bupivacaine, xanthine, aminophylline, sildenafil, tadalafil, vardenafil, udenafil, avanafil, dipyridamole, quinazoline, paraxanthine, papaverine, mesembrenone, rolipram, ibudilast, piclamilast, luteolin, drotaverine, roflumilast, apremilast, crisaborole, inamrinone, milrinone, enoximone, anagrelide, cilostazol, pimobendan, erythro-9-(2-hydroxy-3-nonyl)adenine), (2-[(3,4-dimethoxyphenyl)methyl]-7-[(1R)-1-hydroxyethyl]-4-phenylbutyl]-5-methyl-imidazo[5,1-f][1,2,4]triazin-4(1H)-one), oxindole, (9-(6-phenyl-2-oxohex-3-yl)-2-(3,4-dimethoxybenzyl)-purin-6-one), 3-isobutyl-1-methylxanthine, pentoxifylline, theobromine, triamciclone, tranilast, halofuginone, montelukast, zafirlukast, pirfenidone, nintedanib, theophylline, or a combination thereof.

A 19^{th} aspect of the present disclosure, either alone or in combination with any other aspect herein, concerns the medical device of any of the 1^{st} to 18^{th} aspects, wherein the coating layer further comprises an excipient chosen from a fatty acid, a fatty acid ester, polylactic acid (PLLA, PDLA, PDLLA), polycaprolactone (PCL), sodium docusate, PLGA, PLGA-b-mPEG, polyglutamic acid, polyacrilic acid, hyaluronic acid, alginate, PVA, PVP, Pluronic (PEO-PPO-PEO), cellulose, CMC, HPC, starch, chitosan, human serum albumin (HSA), phospholipids, fatty acid, fatty acid esters, triglycerides, beeswax, cyclodextrin, Tween 20, Tween 80, TPGS, SLS, butylated hydroxytoluene, butylated hydroxyanisole (BHA), vitamin E, vitamin E succinate, and sorbitol esters.

A 20^{th} aspect of the present disclosure, either alone or in combination with any other aspect herein, concerns a medical device comprising a radially-expandable scaffold structure with at least one microneedle positioned circumferentially thereon, wherein the microneedle comprises a coating layer on at least a portion of an outer surface, wherein the coating layer comprises at least one therapeutic agent and wherein the microneedle is part of the radially-expandable scaffold structure.

A 21^{st} aspect of the present disclosure, either alone or in combination with any other aspect herein, concerns the medical device of the 20^{th} aspect, wherein there is no point of connection between the radially-expandable scaffold structure and the at least one microneedle.

A 22^{nd} aspect of the present disclosure, either alone or in combination with any other aspect herein, concerns the medical device of the 20^{th} or 21^{st} aspects, wherein the radially-expandable scaffold structure comprises a stent.

A 23^{rd} aspect of the present disclosure, either alone or in combination with any other aspect herein, concerns the medical device of the 20^{th}, 21^{st}, or 22^{nd} aspects, wherein radially-expandable scaffold structure is adhered to an underlying balloon.

A 24^{th} aspect of the present disclosure, either alone or in combination with any other aspect herein, concerns the medical device of the 20^{th}, 21^{st}, or 22^{nd} aspects, wherein the at least one microneedle is attached to the radially-expandable scaffold structure at a hinge or joint structure thereof.

A 25^{th} aspect of the present disclosure, either alone or in combination with any other aspect herein, concerns the medical device of the 20^{th}, 21^{st}, or 22^{nd} aspects, wherein the at least one microneedle is attached to the radially-expandable scaffold structure along an arm thereof.

A 26^{th} aspect of the present disclosure, either alone or in combination with any other aspect herein, concerns the medical device of the 20^{th}, 21^{st}, or 22^{nd} aspects, wherein the at least one microneedle extends through the radially-expandable scaffold structure from an external surface of an underlying balloon.

A 27^{th} aspect of the present disclosure, either alone or in combination with any other aspect herein, concerns the medical device of the 20^{th}, 21^{st}, or 22^{nd} aspects, wherein the at least one microneedle is of the same material as the radially-expandable scaffold.

A 28^{th} aspect of the present disclosure, either alone or in combination with any other aspect herein, concerns the medical device of any preceding aspect, wherein the at least one microneedle is of biopolymer material, wherein the biopolymer material is selected from the group consisting of poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, modified polyethylene glycol, or a copolymer or mixture thereof and wherein optionally the therapeutic agent is embedded therein.

A 29^{th} aspect of the present disclosure, either alone or in combination with any other aspect herein, concerns the medical device of any of the 20^{th} to 28^{th} aspects, wherein the microneedle is of a metal, wherein the metal is selected from stainless steel, titanium, nickel, tantalum, platinum, palladium, or nitinol.

A 30^{th} aspect of the present disclosure, either alone or in combination with any other aspect herein, concerns the medical device of any of the 20^{th} to 29^{th} aspects, further comprising a hydrophilic-coated thermoplastic layer that covers the coating layer, the radially-expandable scaffold structure, and the at least one microneedle.

A 31^{st} aspect of the present disclosure, either alone or in combination with any other aspect herein, concerns the medical device of the 30^{th} aspect, wherein the hydrophilic-coated thermoplastic layer comprises a biopolymer material selected from the group consisting of poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, modified polyethylene glycol, or a copolymer or mixture thereof.

A 32^{nd} aspect of the present disclosure, either alone or in combination with any other aspect herein, concerns the medical device of the 20^{th}, 21^{st}, or 22^{nd} aspects, wherein the coating layer further comprises nanoparticles with the at least one therapeutic agent loaded therein.

A 33^{rd} aspect of the present disclosure, either alone or in combination with any other aspect herein, concerns the medical device of the 32^{nd} aspect, wherein the nanoparticles comprise a biopolymer selected from poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, modified polyethylene glycol, or a copolymer or mixture thereof.

A 34^{th} aspect of the present disclosure, either alone or in combination with any other aspect herein, concerns the medical device of any of the 20^{th} to 33^{rd} aspects, wherein the therapeutic agent is paclitaxel, rapamycin, daunorubicin, 5-fluorouracil, doxorubicin, bosutinib, ceritinib, crizotinib, gefitinib, ruxolitinib, imatinib, axitinib, nilotinib, trametinib, afatinib, ibrutinib, cabozantinib, imatinib, lenvatinib, sunitinib, regorafenib, sorafenib, vandetanib, dasatinib, pazopanib, Y27632, CA3, verteporfin, VGLL4 peptide, nintedanib, avapritinib, abemaciclib, erdafitinib, fedratinib, palbociclib, pemigatinib, irinotecan, bevasizumab, cetuxamab, biolimus (biolimus A9), everolimus, zotarolimus, tacrolimus, dexamethasone, prednisolone, corticosterone, cisplatin, vinblastine, lidocaine, bupivacaine, xanthine, aminophylline, sildenafil, tadalafil, vardenafil, udenafil, avanafil, dipyridamole, quinazoline, paraxanthine, papaverine, mesembrenone, rolipram, ibudilast, piclamilast, luteolin, drotaverine, roflumilast, apremilast, crisaborole, inamrinone, milrinone, enoximone, anagrelide, cilostazol, pimobendan, erythro-9-(2-hydroxy-3-nonyl)adenine), (2-[(3,4-dimethoxyphenyl)methyl]-7-[(1R)-1-hydroxyethyl]-4-phenylbutyl]-5-methyl-imidazo[5,1-f][1,2,4]triazin-4(1H)-one), oxindole, (9-(6-phenyl-2-oxohex-3-yl)-2-(3,4-dimethoxybenzyl)-purin-6-one), 3-isobutyl-1-methylxanthine, pentoxifylline, theobromine, triamciclone, tranilast, halofuginone, montelukast, zafirlukast, pirfenidone, nintedanib, theophylline, or a combination thereof.

A 35^{th} aspect of the present disclosure, either alone or in combination with any other aspect herein, concerns the medical device of any of the 20^{th} to 34^{th} aspects, wherein the coating layer further comprises an excipient chosen from a fatty acid, a fatty acid ester, polylactic acid (PLLA, PDLA, PDLLA), polycaprolactone (PCL), sodium docusate, PLGA, PLGA-b-mPEG, polyglutamic acid, polyacrilic acid, hyaluronic acid, alginate, PVA, PVP, Pluronic (PEO-PPO-PEO), cellulose, CMC, HPC, starch, chitosan, human serum albumin (HSA), phospholipids, fatty acid, fatty acid esters, triglycerides, beeswax, cyclodextrin, Tween 20, Tween 80, TPGS, SLS, butylated hydroxytoluene, butylated hydroxyanisole (BHA), vitamin E, vitamin E succinate, and sorbitol esters.

A 36^{th} aspect of the present disclosure, either alone or in combination with any other aspect herein, concerns a medical device comprising a radially-expandable scaffold structure with at least one microneedle connected circumferentially thereon, wherein the microneedle comprises a coating layer on at least a portion of an outer surface, wherein the coating layer comprises at least one therapeutic agent.

A 37^{th} aspect of the present disclosure, either alone or in combination with any other aspect herein, concerns the medical device of the 36^{th} aspect, wherein the radially-expandable scaffold structure comprises a stent.

A 38^{th} aspect of the present disclosure, either alone or in combination with any other aspect herein, concerns the medical device of the 36^{th} or 37^{th} aspects, wherein radially-expandable scaffold structure is adhered to an underlying balloon.

A 39^{th} aspect of the present disclosure, either alone or in combination with any other aspect herein, concerns the medical device of the 36^{th} or 37^{th} aspects, wherein the at least one microneedle is adhered or welded to the radially-expandable scaffold structure.

A 40^{th} aspect of the present disclosure, either alone or in combination with any other aspect herein, concerns the medical device of the 36^{th} or 37^{th} aspects, wherein the at least one microneedle is connected to an outer surface of the radially-expandable scaffold structure through a detachable junction.

A 41^{st} aspect of the present disclosure, either alone or in combination with any other aspect herein, concerns the medical device of the 36^{th} or 37^{th} aspects, wherein the at least one microneedle is attached to the radially-expandable scaffold structure at a hinge or joint structure thereof.

A 42^{nd} aspect of the present disclosure, either alone or in combination with any other aspect herein, concerns the medical device of the 36^{th} or 37^{th} aspects, wherein the at least one microneedle is attached to the radially-expandable scaffold structure along an arm thereof.

A 43^{rd} aspect of the present disclosure, either alone or in combination with any other aspect herein, concerns the medical device of the 36^{th} or 37^{th} aspects, wherein the at least one microneedle extends through the radially-expandable scaffold structure from an external surface of an underlying balloon.

A 44^{th} aspect of the present disclosure, either alone or in combination with any other aspect herein, concerns the medical device of any of the 36^{th} to 43^{rd} aspects, wherein the at least one microneedle is of the same material as the radially-expandable scaffold.

A 45^{th} aspect of the present disclosure, either alone or in combination with any other aspect herein, concerns the medical device of any of the 36^{th} to 44^{th} aspects, wherein the at least one microneedle is of biopolymer material, wherein the biopolymer material is selected from the group consisting of poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, modified polyethylene glycol, or a copolymer or mixture thereof and wherein optionally the therapeutic agent is embedded therein.

A 46^{th} aspect of the present disclosure, either alone or in combination with any other aspect herein, concerns the medical device of any of the 36^{th} to 45^{th} aspects, wherein the microneedle is of a metal, wherein the metal is selected from stainless steel, titanium, nickel, tantalum, platinum, palladium, or nitinol.

A 47^{th} aspect of the present disclosure, either alone or in combination with any other aspect herein, concerns the medical device of the 36^{th} or 37^{th} aspects, further comprising a hydrophilic-coated thermoplastic layer that covers the coating layer, the radially-expandable scaffold structure, and the at least one microneedle.

A 48^{th} aspect of the present disclosure, either alone or in combination with any other aspect herein, concerns the medical device of the 47^{th} aspect, wherein the hydrophilic-coated thermoplastic layer comprises a biopolymer material selected from the group consisting of poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, modified polyethylene glycol, or a copolymer or mixture thereof.

A 49^{th} aspect of the present disclosure, either alone or in combination with any other aspect herein, concerns the medical device of the 36^{th} or 37^{th} aspects, wherein the coating layer further comprises nanoparticles with the at least one therapeutic agent loaded therein.

A 50^{th} aspect of the present disclosure, either alone or in combination with any other aspect herein, concerns the medical device of the 49^{th} aspect, wherein the nanoparticles comprise a biopolymer selected from poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, modified polyethylene glycol, or a copolymer or mixture thereof.

A 51^{st} aspect of the present disclosure, either alone or in combination with any other aspect herein, concerns the medical device of any of the 36^{th} to 50^{th} aspects, wherein the therapeutic agent is paclitaxel, rapamycin, daunorubicin, 5-fluorouracil, doxorubicin, bosutinib, ceritinib, crizotinib, gefitinib, ruxolitinib, imatinib, axitinib, nilotinib, trametinib, afatinib, ibrutinib, cabozantinib, imatinib, lenvatinib, sunitinib, regorafenib, sorafenib, vandetanib, dasatinib, pazopanib, Y27632, CA3, verteporfin, VGLL4 peptide, nintedanib, avapritinib, abemaciclib, erdafitinib, fedratinib, palbociclib, pemigatinib, irinotecan, bevasizumab, cetuxamab, biolimus (biolimus A9), everolimus, zotarolimus, tacrolimus, dexamethasone, prednisolone, corticosterone, cisplatin, vinblastine, lidocaine, bupivacaine, xanthine, aminophylline, sildenafil, tadalafil, vardenafil, udenafil, avanafil, dipyridamole, quinazoline, paraxanthine, papaverine, mesembrenone, rolipram, ibudilast, piclamilast, luteolin, drotaverine, roflumilast, apremilast, crisaborole, inamrinone, milrinone, enoximone, anagrelide, cilostazol, pimobendan, erythro-9-(2-hydroxy-3-nonyl)adenine), (2-[(3,4-dimethoxyphenyl)methyl]-7-[(1R)-1-hydroxyethyl]-4-phenylbutyl]-5-methyl-imidazo[5,1-f][1,2,4]triazin-4(1H)-one), oxindole, (9-(6-phenyl-2-oxohex-3-yl)-2-(3,4-dimethoxybenzyl)-purin-6-one), 3-isobutyl-1-methylxanthine, pentoxifylline, theobromine, triamciclone, tranilast, halofuginone, montelukast, zafirlukast, pirfenidone, nintedanib, theophylline, or a combination thereof.

A 52^{nd} aspect of the present disclosure, either alone or in combination with any other aspect herein, concerns the medical device of any of the 36^{th} to 51^{st} aspects, wherein the coating layer further comprises an excipient chosen from a fatty acid, a fatty acid ester, polylactic acid (PLLA, PDLA, PDLLA), polycaprolactone (PCL), sodium docusate, PLGA, PLGA-b-mPEG, polyglutamic acid, polyacrilic acid, hyaluronic acid, alginate, PVA, PVP, Pluronic (PEO-PPO-PEO), cellulose, CMC, HPC, starch, chitosan, human serum albumin (HSA), phospholipids, fatty acid, fatty acid esters, triglycerides, beeswax, cyclodextrin, Tween 20, Tween 80, TPGS, SLS, butylated hydroxytoluene, butylated hydroxyanisole (BHA), vitamin E, vitamin E succinate, and sorbitol esters.

A 53^{rd} aspect of the present disclosure, either alone or in combination with any other aspect herein, concerns a medical device comprising a radially-expandable scaffold structure with at least one microneedle positioned circumferentially thereon, wherein the microneedle comprises a coating layer on at least a portion of an outer surface, wherein the coating layer comprises at least one therapeutic agent and wherein the at least one microneedle extends through the radially-expandable scaffold structure from an external surface of an underlying balloon.

A 54^{th} aspect of the present disclosure, either alone or in combination with any other aspect herein, concerns the medical device of the 53^{rd} aspect, wherein the radially-expandable scaffold structure comprises a stent.

A 55^{th} aspect of the present disclosure, either alone or in combination with any other aspect herein, concerns the medical device of the 53^{rd} or 54^{th} aspects, wherein radially-expandable scaffold structure is adhered to an underlying balloon.

A 56^{th} aspect of the present disclosure, either alone or in combination with any other aspect herein, concerns the medical device of the 53^{rd} or 54^{th} aspects, wherein the at least one microneedle protrudes through the radially-expandable scaffold structure at a hinge or joint structure thereof.

A 57^{th} aspect of the present disclosure, either alone or in combination with any other aspect herein, concerns the medical device of the 53^{rd} or 54^{th} aspects, wherein the at least one microneedle is attached to the radially-expandable scaffold structure along an arm thereof.

A 58^{th} aspect of the present disclosure, either alone or in combination with any other aspect herein, concerns the medical device of any of the 53^{rd} to 57^{th} aspects, wherein the at least one microneedle is of the same material as the radially-expandable scaffold.

A 59^{th} aspect of the present disclosure, either alone or in combination with any other aspect herein, concerns the medical device of any of the 53^{rd} to 58^{th} aspects, wherein the at least one microneedle is of biopolymer material, wherein the biopolymer material is selected from the group consisting of poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, modified polyethylene glycol, or a copolymer or mixture thereof and wherein optionally the therapeutic agent is embedded therein.

A 60^{th} aspect of the present disclosure, either alone or in combination with any other aspect herein, concerns the medical device of any of the 53^{rd} to 59^{th} aspects, wherein the microneedle is of a metal, wherein the metal is selected from stainless steel, titanium, nickel, tantalum, platinum, palladium, or nitinol.

A 61^{st} aspect of the present disclosure, either alone or in combination with any other aspect herein, concerns the medical device of the 53^{rd} or 54^{th} aspect, further comprising a hydrophilic-coated thermoplastic layer that covers the coating layer, the radially-expandable scaffold structure, and the at least one microneedle.

A 62^{nd} aspect of the present disclosure, either alone or in combination with any other aspect herein, concerns the medical device of the 61^{st} aspect, wherein the hydrophilic-coated thermoplastic layer comprises a biopolymer material selected from the group consisting of poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, modified polyethylene glycol, or a copolymer or mixture thereof.

A 63^{rd} aspect of the present disclosure, either alone or in combination with any other aspect herein, concerns the medical device of the 53^{rd} or 54^{th} aspect, wherein the coating layer further comprises nanoparticles with the at least one therapeutic agent loaded therein.

A 64^{th} aspect of the present disclosure, either alone or in combination with any other aspect herein, concerns the medical device of the 63^{rd} aspect, wherein the nanoparticles comprise a biopolymer selected from poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, modified polyethylene glycol, or a copolymer or mixture thereof.

A 65^{th} aspect of the present disclosure, either alone or in combination with any other aspect herein, concerns the medical device of the 53^{rd} to 64^{th} aspects, wherein the therapeutic agent is paclitaxel, rapamycin, daunorubicin, 5-fluorouracil, doxorubicin, bosutinib, ceritinib, crizotinib, gefitinib, ruxolitinib, imatinib, axitinib, nilotinib, trametinib, afatinib, ibrutinib, cabozantinib, imatinib, lenvatinib, sunitinib, regorafenib, sorafenib, vandetanib, dasatinib, pazopanib, Y27632, CA3, verteporfin, VGLL4 peptide, nintedanib, avapritinib, abemaciclib, erdafitinib, fedratinib, palbociclib, pemigatinib, irinotecan, bevasizumab, cetuxamab, biolimus (biolimus A9), everolimus, zotarolimus, tacrolimus, dexamethasone, prednisolone, corticosterone, cisplatin, vinblastine, lidocaine, bupivacaine, xanthine, aminophylline, sildenafil, tadalafil, vardenafil, udenafil, avanafil, dipyridamole, quinazoline, paraxanthine, papaverine, mesembrenone, rolipram, ibudilast, piclamilast, luteolin, drotaverine, roflumilast, apremilast, crisaborole, inamrinone, milrinone, enoximone, anagrelide, cilostazol, pimobendan, erythro-9-(2-hydroxy-3-nonyl)adenine), (2-[(3,4-dimethoxyphenyl)methyl]-7-[(1R)-1-hydroxyethyl]-4-phenylbutyl]-5-methyl-imidazo[5,1-f][1,2,4]triazin-4(1H)-one), oxindole, (9-(6-phenyl-2-oxohex-3-y1)-2-(3,4-dimethoxybenzyl)-purin-6-one), 3-isobutyl-1-methylxanthine, pentoxifylline, theobromine, triamciclone, tranilast, halofuginone, montelukast, zafirlukast, pirfenidone, nintedanib, theophylline, or a combination thereof.

A 66^{th} aspect of the present disclosure, either alone or in combination with any other aspect herein, concerns the medical device of the 53^{rd} to 65^{th} aspects, wherein the coating layer further comprises an excipient chosen from a fatty acid, a fatty acid ester, polylactic acid (PLLA, PDLA, PDLLA), polycaprolactone (PCL), sodium docusate, PLGA, PLGA-b-mPEG, polyglutamic acid, polyacrilic acid, hyaluronic acid, alginate, PVA, PVP, Pluronic (PEO-PPO-PEO), cellulose, CMC, HPC, starch, chitosan, human serum albumin (HSA), phospholipids, fatty acid, fatty acid esters, triglycerides, beeswax, cyclodextrin, Tween 20, Tween 80, TPGS, SLS, butylated hydroxytoluene, butylated hydroxyanisole (BHA), vitamin E, vitamin E succinate, and sorbitol esters.

A 67^{th} aspect of the present disclosure, either alone or in combination with any other aspect herein, concerns a medical device comprising a radially-expandable scaffold structure with at least one microneedle connected circumferentially thereon, wherein the microneedle is comprised of a biopolymer with a therapeutic agent embedded therein.

A 68^{th} aspect of the present disclosure, either alone or in combination with any other aspect herein, concerns the medical device of the 67^{th} aspect, wherein the radially-expandable scaffold structure comprises a stent.

A 69^{th} aspect of the present disclosure, either alone or in combination with any other aspect herein, concerns the medical device of the 67^{th} or 68^{th} aspect, wherein radially-expandable scaffold structure is adhered to an underlying balloon.

A 70^{th} aspect of the present disclosure, either alone or in combination with any other aspect herein, concerns the medical device of the 67^{th} or 68^{th} aspect, wherein the at least one microneedle is adhered or welded to the radially-expandable scaffold structure.

A 71^{st} aspect of the present disclosure, either alone or in combination with any other aspect herein, concerns the medical device of the 67^{th} or 68^{th} aspect, wherein the at least one microneedle is connected to an outer surface of the radially-expandable scaffold structure through a detachable junction.

A 72^{nd} aspect of the present disclosure, either alone or in combination with any other aspect herein, concerns the medical device of the 67^{th} or 68^{th} aspect, wherein the at least one microneedle is attached to the radially-expandable scaffold structure at a hinge or joint structure thereof.

A 73^{rd} aspect of the present disclosure, either alone or in combination with any other aspect herein, concerns the medical device of the 67^{th} or 68^{th} aspect, wherein the at least one microneedle is attached to the radially-expandable scaffold structure along an arm thereof.

A 74^{th} aspect of the present disclosure, either alone or in combination with any other aspect herein, concerns the medical device of the 67^{th} or 68^{th} aspect, wherein the at least one microneedle extends through the radially-expandable scaffold structure from an external surface of an underlying balloon.

A 75^{th} aspect of the present disclosure, either alone or in combination with any other aspect herein, concerns the medical device of any of the 67^{th} to 74^{th} aspects, wherein the at least one microneedle is of the same material as the radially-expandable scaffold.

A 76^{th} aspect of the present disclosure, either alone or in combination with any other aspect herein, concerns the medical device of the 67^{th} or 68^{th} aspect, wherein the at least one microneedle is of biopolymer material, wherein the biopolymer material is selected from the group consisting of poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, modified polyethylene glycol, or a copolymer or mixture thereof and wherein optionally the therapeutic agent is embedded therein.

A 77^{th} aspect of the present disclosure, either alone or in combination with any other aspect herein, concerns the medical device of the 67^{th} or 68^{th} aspect, further comprising a hydrophilic-coated thermoplastic layer that covers the coating layer, the radially-expandable scaffold structure, and the at least one microneedle.

A 78^{th} aspect of the present disclosure, either alone or in combination with any other aspect herein, concerns the medical device of the 77^{th} aspect, wherein the hydrophilic-coated thermoplastic layer comprises a biopolymer material selected from the group consisting of poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, modified polyethylene glycol, or a copolymer or mixture thereof.

A 79^{th} aspect of the present disclosure, either alone or in combination with any other aspect herein, concerns the medical device of the 67^{th} or 68^{th} aspect, wherein medical device further comprises a coating layer comprised of nanoparticles with the therapeutic agent loaded therein.

An 80^{th} aspect of the present disclosure, either alone or in combination with any other aspect herein, concerns the medical device of the 79^{th} aspect, wherein the nanoparticles comprise a biopolymer selected from poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, modified polyethylene glycol, or a copolymer or mixture thereof.

An 81^{st} aspect of the present disclosure, either alone or in combination with any other aspect herein, concerns the medical device of any of the 67^{th} to 80^{th} aspects, wherein the therapeutic agent is paclitaxel, rapamycin, daunorubicin, 5-fluorouracil, doxorubicin, bosutinib, ceritinib, crizotinib, gefitinib, ruxolitinib, imatinib, axitinib, nilotinib, trametinib, afatinib, ibrutinib, cabozantinib, imatinib, lenvatinib, sunitinib, regorafenib, sorafenib, vandetanib, dasatinib, pazopanib, Y27632, CA3, verteporfin, VGLL4 peptide, nintedanib, avapritinib, abemaciclib, erdafitinib, fedratinib, palbociclib, pemigatinib, irinotecan, bevasizumab, cetuxamab, biolimus (biolimus A9), everolimus, zotarolimus, tacrolimus, dexamethasone, prednisolone, corticosterone, cisplatin, vinblastine, lidocaine, bupivacaine, xanthine, aminophylline, sildenafil, tadalafil, vardenafil, udenafil, avanafil, dipyridamole, quinazoline, paraxanthine, papaverine, mesembrenone, rolipram, ibudilast, piclamilast, luteolin, drotaverine, roflumilast, apremilast, crisaborole, inamrinone, milrinone, enoximone, anagrelide, cilostazol, pimobendan, erythro-9-(2-hydroxy-3-nonyl)adenine), (2-[(3,4-dimethoxyphenyl)methyl]-7-[(1R)-1-hydroxyethyl]-4-phenylbutyl]-5-methyl-imidazo[5,1-f][1,2,4]triazin-4(1H)-one), oxindole, (9-(6-phenyl-2-oxohex-3-yl)-2-(3,4-dimethoxybenzyl)-purin-6-one), 3-isobutyl-1-methylxanthine, pentoxifylline, theobromine, triamciclone, tranilast, halofuginone, montelukast, zafirlukast, pirfenidone, nintedanib, theophylline, or a combination thereof.

An 82^{nd} aspect of the present disclosure, either alone or in combination with any other aspect herein, concerns the medical device of the 79^{th} aspect, wherein the coating layer further comprises an excipient chosen from a fatty acid, a fatty acid ester, polylactic acid (PLLA, PDLA, PDLLA), polycaprolactone (PCL), sodium docusate, PLGA, PLGA-b-mPEG, polyglutamic acid, polyacrilic acid, hyaluronic acid, alginate, PVA, PVP, Pluronic (PEO-PPO-PEO), cellulose, CMC, HPC, starch, chitosan, human serum albumin (HSA), phospholipids, fatty acid, fatty acid esters, triglycerides, beeswax, cyclodextrin, Tween 20, Tween 80, TPGS, SLS, butylated hydroxytoluene, butylated hydroxyanisole (BHA), vitamin E, vitamin E succinate, and sorbitol esters.

These and additional features provided by the aspects described herein will be more fully understood in view of the following detailed description, in conjunction with the drawings.

### BRIEF DESCRIPTION OF THE DRAWINGS

The aspects set forth in the drawings are illustrative and exemplary in nature and not intended to limit the subject matter defined by the claims. The following detailed description of the illustrative aspects can be understood when read in conjunction with the following drawings, where like structure is indicated with like reference numerals and in which:

FIG. 1 is a schematic of an embodiment of the disclosure.

FIG. 2 is a schematic of a further embodiment of the disclosure.

FIG. 3 is a schematic of an even further embodiment of the disclosure.

FIG. 4A is a side view of an embodiment of the disclosure.

FIG. 4B is a head on view of the embodiemtn of FIG. 4A.

FIG. 5 is a side view of an embodiment of the disclosure.

FIG. 6A is a head on view of an embodiment of the disclosure.

FIG. 6B is an angled of the embodiment of FIG. 6A.

FIG. 7A is a head on view of an embodiment of the disclosure.

FIG. 7B is an angled of the embodiment of FIG. 7A.

FIG. 8A is a head on view of an embodiment of the disclosure.

FIG. 8B is an angled of the embodiment of FIG. 8A.

FIG. 9 is a schematic of an embodiment of the disclosure.

FIG. 10 is a schematic of a further embodiment of the disclosure.

FIG. 11 is a schematic of an even further embodiment of the disclosure.

FIG. 12 is a schematic of a fourth embodiment of the disclosure.

### DESCRIPTION

The present disclosure concerns one or more microneedles coated and/or encapsulated with a therapeutic agent on an exterior surface of a medical device that is radially expandable when placed in situ, such as a stent or a balloon. The microneedles are positioned on the exterior surface in a manner such that when the vascular device is expanded radially in situ, the microneedle can puncture the vessel wall. The microneedle, however, is of a limited depth or height perpendicular to the vascular device such that it will not reach or breach the tunica adventitia of the vessel. In some aspects, the microneedle is of a depth or height that it will reach or pass through the tunica intima when deployed. In some aspects, the microneedle is of sufficient depth or height to reach the tunica media of a vessel wall. It will be appreciated that the depth of the puncture within the vessel wall correlates to the perpendicular height of the microneedle above the outer surface of the medical device when expanded, as the outer surface is designed to touch or press against the vessel wall when deployed. Accordingly, reference to microneedle length is with respect to a microneedle positioned with the point perpendicular to the outer surface and concerns the distance between the point of the microneedle and the point of contact with the outer surface of the medical device. It will also be appreciated that in some instances a subject's vessel wall may not fully penetrate due to resistance such as with calcified deposits. It will still be appreciated that the microneedles as described herein may score and/or crack such tissue. Accordingly, it is to be understood that as described herein, reference to puncturing the vessel wall may include scoring, scratching, cracking, or roughing the vessel wall or deposits thereon.

In some aspects, the present disclosure concerns a medical device with a radially-expandable scaffold structure and at least one microneedle positioned circumferentially thereon. In some aspects, the radially-expandable scaffold structure is a collapsible device such that it can be threaded through an opening or vessel in a subject and then expand once in situ through the control of a user. In some aspects, the medical device can expand radially through the inflation of a balloon located within the collapsed structure. It will be appreciated that the microneedle or microneedles are of a minimal perpendicular height from the collapsed structure such that the overall circumference of the collapsed structure does not significantly increase and the medical device can retain its ability to be successfully maneuvered into a desired location without injury. In some aspects, the microneedle or microneedles are positioned on the medical device in a manner that allows the point of the microneedle to not protrude from the collapsed structure or to point inward or at an angle less than perpendicular from the collapsed structure. In such aspects, as the scaffold expands, the microneedle point rotates relative to the overall circumference and becomes perpendicular when the scaffold is fully-expanded. In some aspects, the radially-expandable scaffold structure includes a stent, is part of a stent, or is a stent. In some aspects, the stent is adhered to the balloon.

In some aspects, one or more of the microneedles can be positioned over a hinge or a joint of two or more arms of the scaffold structure. In some aspects, one or more microneedles are positioned along the exterior of an arm of the scaffold structure.

In some aspects, the present disclosure relates to microneedles on the surface of a stent. In some aspects, the stent is an implantable stent. In some aspects, the stent is a scaffold around a balloon or set within the interior thereof. In other aspects, the microneedles are on the exterior surface of a balloon. It will be apparent that in some aspects, a microneedle can be affixed to the exterior surface of a vascular device. It will also be apparent that in some aspects, the microneedle can exist as a protrusion from the exterior surface of the medical device that is formed or shaped during the manufacture thereof.

In some aspects, the microneedle includes a sharp point at the distal end to puncture the vessel wall. The sharp point tapers outwardly on at least one side of the microneedle as it moves toward the proximal end on the exterior surface of the medical device. In some aspects, the microneedle includes, at least in part, a conical shape. It will be appreciated, however, that a circular perimeter is not required to ensure that the microneedle includes a sharp point at the distal end to puncture the vessel.

In some aspects, the microneedle is of the same material as the underlying medical device. In some aspects, the microneedle is formed as part of the medical device. In other aspects, the microneedle is attached after manufacturing of the underlying medical device, such as through the inclusion of an adhesive or weld between the microneedle's proximal end and the exterior surface of the medical device. In some aspects, the microneedle is of a material with sufficient rigidity that the point at the distal end will not flex when the medical device is expanded radially and the point comes into contact with the vessel wall as it occupies the space within the lumen, allowing the point to puncture the wall of the vessel. In some aspects, the microneedle is of a metal material, such as steel or titanium or other medically or immunologically inert metal. It will be appreciated that the metal material should be resistant to oxidation or corrosion and be non-reactive to any sterilization process or cause any adverse reaction within the human body. In some aspects, the microneedle is of a polymer. In some aspects, the microneedle is nitinol or a nickel titanium composite or alloy.

In some aspects, the microneedle is of a biopolymer material or a combination or composite thereof. In some aspects, such materials may include poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, and modified polyethylene glycol, as well as copolymers and/or mixtures thereof. In some aspects, the microneedle is formed by casting the biopolymer in a mold.

In some aspects, the microneedle is of a biodegradable polymer, such that the polymer will dissolve and/or be absorbed by the subject over a period of time. It will be appreciated that the choice of biopolymer(s), as well as the concentrations(s) thereof can determine the rate at which the microneedle will degrade within the subject.

In some aspects, the present disclosure may include the incorporation of one or more therapeutic agents within a biopolymer microneedle. In some aspects, the microneedle may include nanoparticles as described herein that include a therapeutic. In some aspects, the nanoparticles may be coated on the microneedle. In some aspects, the nanoparticles may be embedded within the microneedle. In some aspects, the nanoparticles may be both coated on the microneedle and embedded therein. In some aspects, the microneedle may be embedded with a combination of therapeutic and nanoparticles embedded with a therapeutic. It will be appreciated that in some aspects, the therapeutic agents may be the same, yet in others, they may differ in composition and/or concentration and/or combination with other therapeutic agents. It will be appreciated that through the combination of options of the biopolymer, the coating layer and the nanoparticles, that the microneedles of the present disclosure can provide a desired release profile over a desired time course, as well as providing multiple different therapeutics also available at the same or different release profiles. It will also be appreciated that multiple types of microneedle may be deployed across the same stent, such that one microneedle contains on type of biopolymer and a therapeutic and another microneedle is of a second biopolymer with the same or a different therapeutic therein. Such examples may also include a combination of microneedles where some are of one or more biopolymers and others are of a metal. It may also be appreciated that microneedle depth can be varied as well to deliver therapeutics to different regions or zones within the vessel wall or tissue or deposits thereof.

In some aspects, the microneedle cannot be removed or easily dislodged from the surface of the medical device. For example, in instances where the medical device is deployed on a permanent, semi-permanent, or prolonged basis, such as a stent, the microneedle can puncture and remain embedded in the tunica media.

In some aspects, the microneedle is arranged on the surface of the medical device in a manner that allows the microneedle to separate from the medical device, such that when the medical device is expanded radially, the microneedle punctures and embeds within the tunica media. As the device contracts radially, the microneedles detach from the medical device and remain embedded in the tunica media. In such instances, the microneedle can detach either by overcoming an adhesive force between the exterior of the medical device and the proximal end of the microneedle or by including a fracture or break-point between the exterior surface of the medical device and the proximal end of the microneedle, such that once embedded in the tunica media, the microneedle detaches from the medical device. In some aspects, an adhesive with low adhesion is utilized to retain the microneedles on the exterior surface of the medical device until it is deployed in situ. In some aspects, an adhesive that dissolves or deteriorates in situ is utilized to retain the microneedle. In some aspects, the microneedle is affixed to the medical device with a biocompatible adhesive. In some aspects, the microneedle may be connected to the underlying medical device through a weld.

In some aspects, the microneedle may be integrally formed with the medical device or cast as an original part of the medical device. For example, in manufacturing of the parts of the medical device, such as the joints or hinges and the arms connecting such, microneedles may be included in the mold or casting mechanism, such that the microneedles are indiscernible from the rest of the medical device and/or that there is no point of connection between the microneedle and the rest of the medical device.

In some aspects, the microneedle includes a coating layer on at least a portion of an exterior surface thereof. In some aspects, a coating layer may be on the exterior surface of the medical device. In some aspects, a coating layer may be on both the exterior surface of the medical device and the exterior of the microneedles. In some aspects, the coating layer on the exterior of the medical device may differ from the coating layer on the exterior of the microneedle. In some aspects, the coating layers are the same, or at least include similar components therein. In some aspects, there may be more than one coating layer on the exterior of the microneedles and/or the exterior of the medical device. The coating layer may provide one or more beneficial aspects to the tunica media once the microneedle has punctured therein. In some aspects, the microneedle is coated with a therapeutic agent. In some aspects, the coating layer is applied to the outer surface of the microneedles and/or medical device to allow and/or provide contact between the drug coating layer and the inner walls of vasculature vessel or the walls defining a lumen. As identified herein, the medical devices are for implantation and/or insertion within a vessel's lumen of the vasculature or circulatory system of a subject. By application of a drug coating layer to the outer or exterior surface of the medical device, the drug coating layer is able to come into contact with the inner surface of a vessel or lumen wall at one or more points. In some aspects, one or more drug coating layer(s) of the present disclosure may come into contact with the vessel wall when the device is expanded within the vessel of the subject. In some aspects, providing the coating layer to the external surface of the microneedle will allow for the therapeutic agent(s) within the coating layer to transfer to and/or be absorbed by the interior of the vessel, such as though the tunica media. In further aspects, the coating layer may be of one or more layers of either the same layer or of mixed compositions. Those skilled in the art will appreciate that interior layers to an outer coating layer may come into contact with the inner walls of a vessel as a preceding outer layer is removed and/or disintegrates to expose such to the inner vessel wall.

In some aspects, the microneedle is of a metal or polymer material. In some aspects, the polymer materials are selected on the basis that they are biodegradable and/or bioabsorbed by the body over time. As used herein, “bioabsorbable” refers to a compound that can be absorbed by the surrounding or local tissue of a subject and/or degraded and absorbed by the tissue of the subject.

In some aspects, the microneedle is of a biodegradable polymer material. In some aspects, the biodegradable polymer material is a biocompatible polymer. In some aspects, the biodegradable polymer material may be coated with a coating layer as set forth herein. In some aspects, the coating layer provides a therapeutic agent(s) to the medical device, such that when the microneedle encounters and/or punctures the vessel, the therapeutic is transferred to and/or absorbed by the subject.

In some aspects, as the biodegradable polymer itself may include at least one or more therapeutic agents. In some aspects, the microneedle is encapsulated with a therapeutic agent. Such microneedles can be prepared by adding one or more therapeutics to a polymer solution prior to cross-linking. In such aspects, the microneedle may be of a biodegradable material, such that as the biodegradable material erodes, the therapeutic agents are released and can be absorbed by the subject.

In some aspects, the present disclosure concerns microneedles arranged on the periphery or external surface of a stent, such that the microneedles can penetrate the vessel wall or deposits thereon when expanded in situ. In some aspects, the microneedles are of a metal and/or a bioabsorbable/biodegradable polymer. In some aspects, the needles are coated with a coating layer that includes a therapeutic. In some aspects, the needles themselves may be of a biopolymer material that itself is loaded or embedded with one or more therapeutics. In some aspects, the needles extend through a stent from an underlying balloon. In some aspects, an additional hydrophilic layer covers the collapsed stent. It some aspects, the needles detach from the stent, and in other aspects, the needles do not or are formed as part of the stent itself. It will be appreciated that in discussing each of the aspects individually that they may be combined with any one or more additional features discussed herein.

### Microneedles

In some aspects, the present disclosure concerns microneedles of a metal and/or biopolymer material. In some aspects, the microneedle is of a metal material, such as steel or titanium or other medically or immunologically inert metal, as well as non-reactive to any sterilization process or cause any adverse reaction within the human body. In some aspects, the microneedle is nitinol or a nickel titanium composite or alloy.

In some aspects, the microneedle is of a biopolymer material or a combination or composite thereof. In some aspects, such materials may include poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, and modified polyethylene glycol, as well as copolymers and/or mixtures thereof. In some aspects, the microneedle is formed by casting the biopolymer in a mold.

In some aspects, the microneedle is integrally formed with or formed as part of the medical device. In other aspects, the microneedle is adhered or welded to the external surface of the medical device. In some aspects, the microneedle is attached to the surface of an underlying balloon and is threaded through apertures in an overlying stent such that when the balloon inflates in situ, the needle expands with the stent and pierces the vessel or surrounding tissue or deposit.

In some aspects, the microneedles are designed to detach from the external surface of the medical device. In some aspects, as described herein, therapeutics may be coated and/or embedded in the microneedles. As such, allowing the microneedles to detach allows for them to embed and remain within the vessel wall and deliver therapeutics over a prolonged period of time. In aspects where the microneedles are of a biopolymer, as the microneedle degrades, more therapeutic may be released until the microneedle is completely absorbed by the subject.

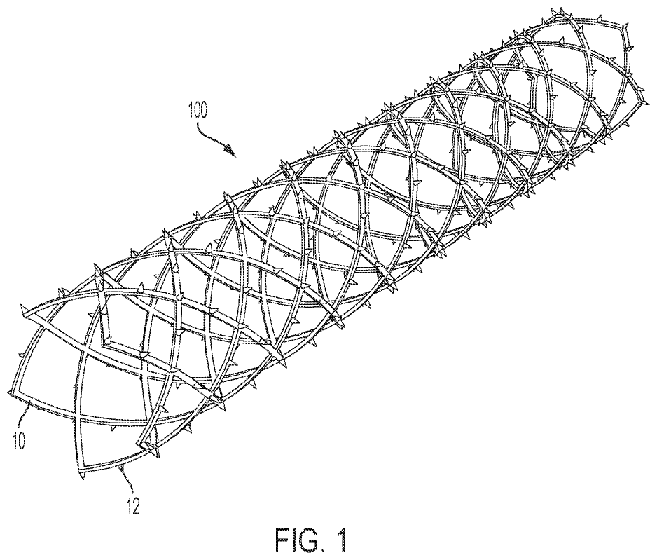

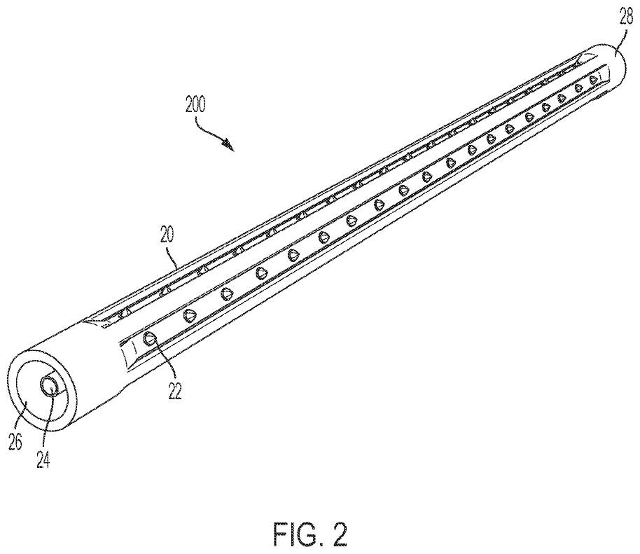

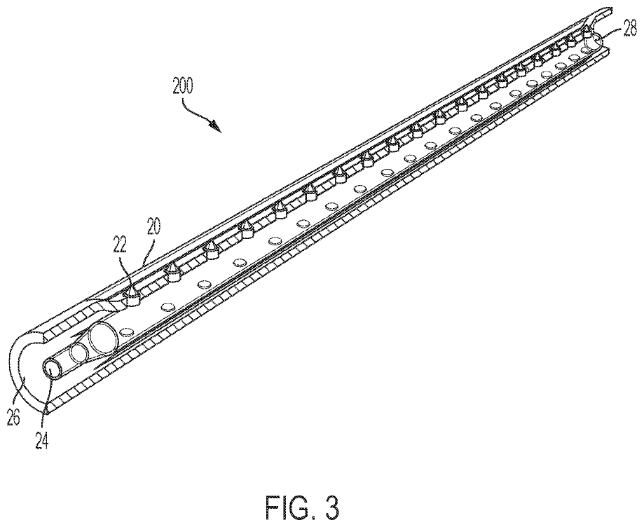

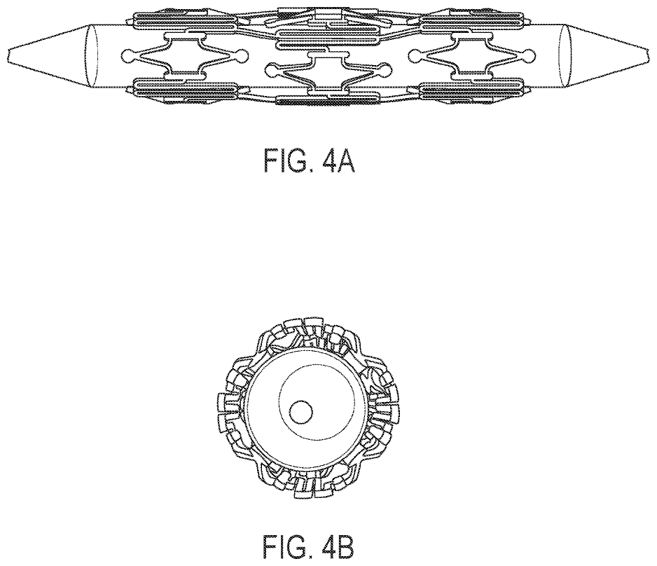

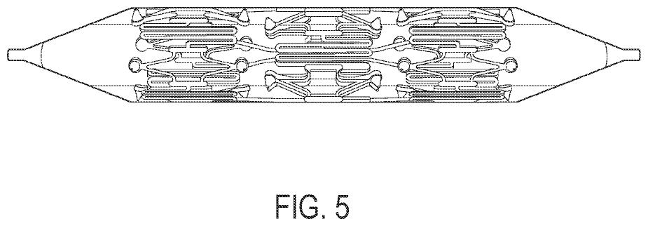

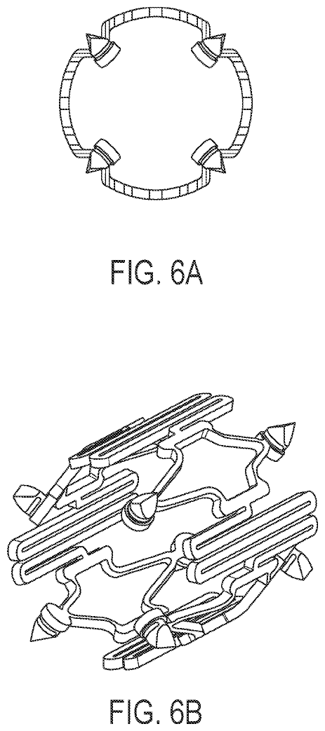

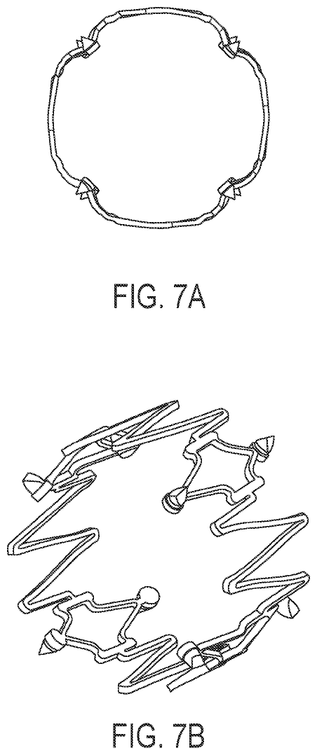

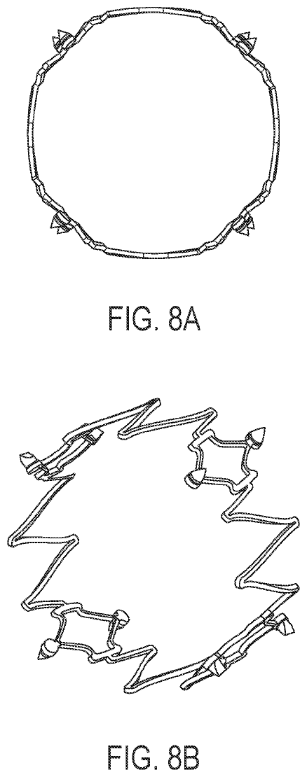

FIGS. 1-8 depict various embodiments of the present disclosure. FIG. 1 shows an expanded stent 100 with microneedles 12 dispensed about the stent arms 10. FIGS. 2 and 3 shows a stent 200 of a stent body 28, with the microneedles 22 recessed therein. The microneedles 22 are on the exterior surface of the balloon 24. The balloon 24 pushes out the inner wall 26, causing the microneedles 22 to project outward (not depicted). FIG. $A shows a further embodiment of the collapsed stent without microneedles. FIG. 4B shows the head on view. FIG. 5 shows the same side view as FIG. 4A with microneedles attached at the joints of the stent arms. FIG. 6A depicts an embodiment where the microneedle points do not protrude from the stent in the collapsed state, but project when expanded, as see in FIG. 6B. FIGS. 7A, 7B, 8A, and 8B show slightly different configurations to 6A and 6B that achieve similar outcomes in the collapsed and expanded states.

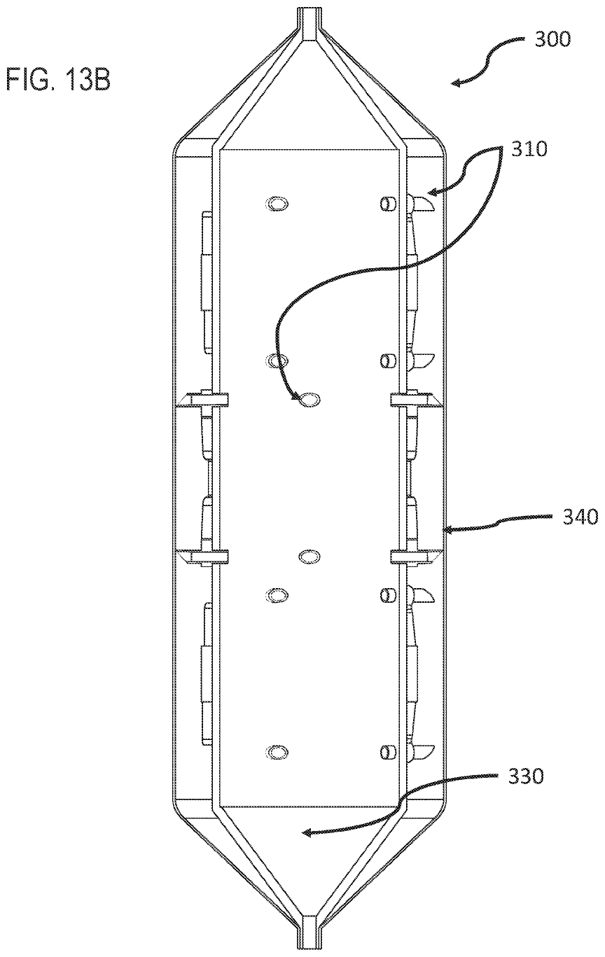

In FIG. 11, a medical device 100 is depicted of a stent 120 surrounding an underlying balloon 130. Dispersed around the exterior of the stent 120 are microneedles 110. As depicted, the microneedles 110 may overlay or be part of a joint or hinge within the stent 120, though such is not required. As the balloon 130 expands in situ, the stent 120 similarly expands by arms moving radially away from the joints or hinges. As the stent 120 expands with the balloon 130 in situ, the point of the microneedles 110 will push against the vessel wall or material deposited therein. With increased pressure in the balloon 130, the microneedles 110 are forced into piercing the surrounding tissue. In some aspects, the microneedles 110 become lodged in the vessel wall, such that with deflation of the balloon, the stent 120 and the microneedles 110 remain. In other aspects, the microneedles 110 detach from the stent 120 and remain embedded in the vessel wall.

### Hydrophilic Coating

In some aspects, the present disclosure concerns a hydrophilic thermoplastic coating layer applied over the external surface of the medical device. In some aspects, the hydrophilic thermoplastic coating layer covers the microneedles and the scaffold body of the medical device. In some aspects, the hydrophilic thermoplastic coating layer covers at least the microneedles. In some aspects, the hydrophilic thermoplastic coating layer covers at least the scaffold structure. In some aspects, the hydrophilic thermoplastic coating layer covers the coating layer(s) as set forth herein.

In some aspects, the hydrophilic coating covers the collapsed scaffold, protecting any coatings or surfaces therein from exposure to the turbulence of the subject's circulation as the device is moved into place. In some aspects, the hydrophilic coating may dissolve or disintegrate allowing the underlying scaffold and microneedles to be exposed to the circulatory system. In some aspects, the hydrophilic coating may tear or be pierced as the scaffold expands once in situ. It will also be appreciated that the hydrophilic coating may adhere or be attracted to the vessel wall due to the physical nature of the layer. In some aspects, the hydrophilic coating is of a hydrophilic biopolymer as set forth herein.

Turning to FIG. 12, a medical device 200 is depicted with a stent 220 and an underlying balloon 230 and microneedles 210 dispersed on the outer surface of the stent 220. A hydrophilic layer 240 surrounds the entire device. When the balloon 230 expands, the stent 220 and microneedles 210 expand outwardly and the microneedles 220 will pierce the hydrophilic coating as they similarly push into the vessel walls in situ, Further, by being of a hydrophilic material, the hydrophilic layer 240 can adhere to the vessel wall. In aspects where the hydrophilic layer 240 is of a bioabsorbable biopolymer material, the hydrophilic layer 240 can be degraded and/or absorbed when placed in situ within a subject.

In further aspects, the drug-containing coating layer(s) may be further covered by a covering layer to protect the coating layer(s) until the medical device is in the desired location within the subject. In some aspects, the covering layer can be of a water-soluble material such that the layer can dissolve within the flow of the circulatory system of the subject and expose the drug-containing coating layer(s). The presence of the covering layer allows for the drug coating layer to remain protected or covered or partially covered as the medical device is moved to a desired location in the subject, thereby avoiding unnecessary or unintended deposit of the therapeutic agent to a part of a vessel wall or vasculature away from the desired site of treatment. It will be appreciated that the covering layer can be of a varying thickness to allow for sufficient transport time to position the medical device to its desired location in situ. It will also be appreciated that the medical device may be left or maintained in position before allowing expansion to allow for the covering layer to dissolve. In other aspects, it will be appreciated that allowing the medical device to incubate within the subject during the positioning of the medical device and optionally once the medical device is in place to allow the drug-coating matrix to reach sufficient temperature to become tacky or sticky. It will be appreciated that a longer incubation period may provide for improved tackiness of the drug-coating matrix layer.

In some aspects, once the medical device coated with at least two coatings is exposed to blood, the water-soluble overlying covering layer is dissolved, and only the drug coating layer remains. With the drug coating layer shifting to a glass transition phase in situ, the underlying drug coating layer is tacky with respect to the vessel wall. In further aspects, the hydrophobic nature of the drug coating layer prevents the drug coating layer from being washed away by blood and reduces drug loss during delivery. Further, since glass transition temperature of the drug coating layer is lower than the body temperature, the drug coating layer becomes tacky and sticky in situ, allowing the drug coating to be pressure sensitive and adhere to the vessel wall when the medical device is expanded therein. For example, when a balloon is inflated in situ and the drug coating layer comes into contact with the vessel wall, the drug coating layer is pressed against the vessel wall and easily transferred to the vessel wall. The hydrophobic nature of the drug coating layer can additionally provide good adhesion between the drug coating layer and the tissue of the lumen of the vessel.

In some aspects, the water soluble outer covering layer is of a powder, granules or film either adhered to or secured to the medical device such that it covers, entirely or partially, the underlying drug-containing coating layer(s). In some aspects, the water soluble covering layer may be partially embedded within the underlying drug-containing coating layer to cover the therapeutic embedded therein.

The water soluble covering layer itself can be of any non-toxic water soluble compound or combination thereof. Such may include water-soluble salts, water-soluble carbohydrates (e.g., monosaccharides, disaccharides, oligosaccharides and polysaccharides), and/or water-soluble polymers. By way of example, water soluble salts may include, but are not limited to, sodium salts, potassium salts, ammonium salts, nitrate salts, chloride salts, and sulphate salts. Suitable carbohydrates may include, but are not limited to, sorbitol, mannitol, sugar alcohols, fructose, glucose, galactose, sucrose, lactose, maltose, starch, dextrin, cellulose, pectin and glycogen. Water-soluble polymers may include, but are not limited to, polyethyleneglycol, polyvinyl pyrrolidone, polyvinyl alcohol, polyacrylic acid, polyacryl amides, chitosan, phosphoproteins, casein sodium, casein, dextran, hyaluronic acid, and/or albumin Balloon Microneedles In some aspects, the microneedles may be, at least in part, extensions from an underlying balloon that extend through a stent, such that when the balloon expands, the microneedles dispersed on the exterior surface thereof push outward along with the expansion of the stent. In some aspects, the microneedles are adhered to the exterior surface of the balloon. In some aspects, the adhesion may fail when in situ. In other aspects, the microneedles remain adhered. In some aspects, the microneedles are part of the balloon. For example, when the balloon is extruded from a polymer, the mold includes regions such that solid microneedles of the same polymer are deposited on the exterior surface.

Turning to FIG. 13A depicted is a medical device 300 that includes a balloon 330 and a surrounding stent 320. FIG. 13B shows the same medical device 300 absent the stent 320. Microneedles 310 protrude from the exterior surface of the balloon 330 and through the stent 320. As the balloon 330 expands in situ, the microneedles 310 push radially outward and into the surrounding tissue.

### Therapeutics

In some aspects, the microneedles are of a degradable material, such that when deployed within the vessel walls of a subject, the microneedles erode over time and the materials thereof are absorbed and/or metabolized by the subject. In some aspects, the microneedles are of a biopolymer as described herein. In some aspects, the rate of degradation of the microneedle is determined by the selection of biopolymer(s), as well as the concentration therein. For example, some biopolymers are understood to more rapidly degrade within a subject. Similarly, a higher concentration of biopolymer can slow the rate of bioabsorption, as can a higher degree of cross-linking therein.

In some aspects, it can be of benefit to include one or more therapeutics in the formulation of the formed microneedle. In some aspects, nanoparticles embedded with one or more therapeutics may be included in the formulation to prepare the microneedles. In either case, either with therapeutic or nanoparticles embedded within the microneedle, the release of such into the subject and the subject's vessel wall is controlled by the degradation of the biopolymer of the microneedle, thereby providing a controlled and/or sustained release of therapeutic and/or nanoparticle. In aspects where nanoparticles are included, the degradation of the same provides a further mechanism by which therapeutic release may be controlled and/or sustained within a subject over a desired time frame.

In some aspects, the microneedles and/or exterior surface may include a coating layer at least partially thereon. In some aspects, the coating layer on the microneedle and/or medical device includes one or more therapeutic agents. In some aspects, the therapeutic may be directly in the coating layer. In some aspects, the therapeutic may be included within the body of a microparticle. Microparticles may be prepared the evaporation of a solvent with a bioabsorbable/biodegradable polymer and at least one therapeutic therein. In some aspects, the solvent is of dichloromethane (DCM) or ethyl acetate (EtOAc). Polymers may include a network of a poly-glycolic acid (PGA) and a poly-L-lactic acid (PLLA). Other bioabsorbable polymers that can be utilized in combination or alone for the microparticles include polycaprolactone (PCL), poly-DL-lactic acid (PDLLA), poly(trimethylene carbonate) (PTMC), poly (ester amine)s (PEA), poly(para-dioxanone) (PPDO), poly-2-hydroxy butyrate (PHB), and co-polymers with various ratios thereof. In some aspects, the bioabsorbable polymer may include, either alone or in combination with other bioabsorbable polymers, a polymer combination of lactic acid and glycolic acid, poly-lactic-co-glycolic acid (PLGA). Those skilled in the art will appreciate that PLGA can be of varying percentages of lactic acid and glycolic acid, wherein the higher the amount of lactide units, the longer the polymer can last in situ before degrading. Additional tunable properties with PLGA concern the molecular weight, with higher weights showing increased mechanical strength. In some aspects, the polymer microparticle is also loaded or embedded with an antioxidant, such as butylated hydroxytoluene (BHT).

The therapeutic utilized in the microneedles and/or coating layer(s) of the medical device according to some aspects includes a therapeutic agent and at least one additive. In some aspects, the therapeutic may include a cytostatic agent, an anti-fibrosis drug, a macrolide, a kinase inhibitor, a cytotoxic agent, a phosphodiesterase inhibitor, or combinations thereof, which may be viable targets for the treatment of restenosis with improved specificity and less adverse effects.

In some aspects, the therapeutic may include one or more of paclitaxel, sirolimus (rapamycin), daunorubicin, 5-fluorouracil, doxorubicin, sunitinib, sorafenib, irinotecan, bevacizumab, cetuxamab, biolimus (biolimus A9), everolimus, zotarolimus, tacrolimus, tadalafil, sildenafil, dexamethasone, prednisolone, corticosterone, 5-fluorouracil, cisplatin, vinblastine, lidocaine, bupivacaine, and all analogs, derivatives, isomers, racemates, diastereoisomers, prodrugs, hydrates, esters, and/or analogs thereof.

In certain aspects, the therapeutic can be a cytostatic agent, such as a limus drug. A limus drug may include one or more of sirolimus, biolimus (biolimus A9), everolimus, zotarolimus, and tacrolimus. In certain aspects, the therapeutic present in the microneedle and/or drug coating layer, directly and/or through loading in a polymer microparticle, is of from about 1 μm to about 20 μm.

In some aspects, the coating layer may include a polymer coating, such as a biopolymer as set forth herein. In some aspects, the polymer coating may be of a ratio of embedded therapeutic agent or polymer microparticle to polymer coating of from about 0.01 to about 100, including 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 99 and any number therein. In some aspects, the therapeutic agent to polymer ratio may be of from about 0.1 to about 10. In some aspects, the therapeutic agent may be a crystalline therapeutic agent.

In some aspects, the therapeutic agent or polymer microparticle may be provided within the polymer coating at a certain density on the coating layer of medical device. In some aspects, the therapeutic agent may be a crystalline therapeutic agent. In some aspects, the density of the therapeutic agent or polymer microparticle within the polymer coating is of from about 0.1 to 10 μg/mm². In certain aspects, the therapeutic agent or polymer microparticle is provided on the device in the polymer coating at a density of from about 0.5 to about 5 μg/mm². In some aspects, the dose density of the therapeutic agent(s) on the medical device and/or within each polymer microparticle can vary from about 0.1 to about 10 μg/mm², including about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8., 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, and 9.9 μg/mm². In some aspects, the drug dose density is of about 0.5 to about 5 μg/mm².

In some aspects, the concentration density of the therapeutic agent in the drug coating or within the polymer microparticle may be from 0.1 μg/mm² to 10 μg/mm², from 0.1 μg/mm² to 8 μg/mm², from 0.1 μg/mm² to 6 μg/mm², from 0.1 μg/mm² to 4 μg/mm², from 0.1 μg/mm² to 2 μg/mm², from 0.1 μg/mm² to 1 μg/mm², from 1 μg/mm² to 10 μg/mm², from 1 μg/mm² to 8 μg/mm², from 1 μg/mm² to 6 μg/mm², from 1 μg/mm² to 4 μg/mm², from 1 μg/mm² to 2 μg/mm², from 2 μg/mm² to 10 μg/mm², from 2 μg/mm² to 8 μg/mm², from 2 μg/mm² to 6 μg/mm², from 2 μg/mm² to 4 μg/mm², from 4 μg/mm² to 10 μg/mm², from 4 μg/mm² to 8 μg/mm², from 4 μg/mm² to 6 μg/mm², from 6 μg/mm² to 10 μg/mm², from 6 μg/mm² to 8 μg/mm², or from 8 μg/mm² to 10 μg/mm². In some aspects the concentration density of the at least one therapeutic agent in the drug coating or polymer microparticle may be from 0.5 μg/mm² to 5 μg/mm².

The methods to apply the coating layer include to a medical device may include dip coating, metering coating, spray coating, electrostatic spray coating, roller coating, spin coating, ink-jet printing, 3D printing, or combinations thereof. A preferred method is metering coating and spray coating. After the solvent has evaporated, the coating layer is left on the surface.

In some aspects, the coating layer may include a crystalline therapeutic agent and/or an amorphous therapeutic agent of a particular size range or ranges. In some aspects, the crystalline and/or amorphous therapeutic agent can be embedded within the coating layer. In other aspects, the crystalline and/or amorphous therapeutic agent is loaded within a polymer microparticle embedded in the coating layer. In further aspects, the crystalline and/or amorphous therapeutic agent adheres to the surface of the medical device through the evaporation of a solvent. In some aspects, the crystalline and/or amorphous therapeutic agent microparticle size can vary from about 0.1 μm to about 100 μm, including about 0.2, 0.3, 0.4, 0.5, 0.6, 0.7, 0.8, 0.9, 1.0, 2, 3, 4, 5, 6, 7, 8, 9, 10, 20, 30, 40, 50, 60, 70, 80, 90, 99 μm and any size or number therein. In some aspects, the particle size is of from about 1 μm to about 20 μm. In other aspects, the particle size of from about 10 μm to about 100 μm. Size selection can be achieved through methods understood in the art, such as by passing through mesh of a pre-determined pore or hole size. The desired particle size can be achieved by dry grind or wet grinding. The grinding method may include techniques such as use of a jaw crusher, ultra-centrifugal mill, cyclone mill, cross beater mill, rotor beater mill, cutting mill, knife mill, mortar grinder, disc mill, mixer mill, cryomill, planetary ball mill, drum mill, and/or fine grinding rod mill. In some aspects, the particle size may be achieved with use of a ball mill. The ground drug particles and polymer mix may be combined with a solvent (or a mixture of solvents) and form a slurry coating solution. The methods may also include application of the slurry coating solution to a medical device surface. Such techniques for application may include dip coating, metering coating, spray coating, electrostatic spray coating, roller coating, spin coating, ink-jet printing, and 3D printing. In certain aspects, the method includes metering coating.

As used herein, “derivative” refers to a chemically or biologically modified version of a chemical compound that is structurally similar to a parent compound and (actually or theoretically) derivable from that parent compound (for example, dexamethasone). A derivative may or may not have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al., Advanced Drug Delivery Reviews 19 (1996) 115; Design of Prodrugs, H. Bundgaard (ed.), Elsevier, 1985; or H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form alkali metal salts or alkaline earth metal salts (e.g., sodium salts, potassium salts, magnesium salts and calcium salts, as well as salts with physiologically tolerable quaternary ammonium ions and acid addition salts with ammonia and physiologically tolerable organic amines such as triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds which simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange.

As used herein, “analog” or “analogue” may refer to a chemical compound that is structurally similar to another but differs slightly in composition (as in the replacement of one atom by an atom of a different element or in the presence of a particular functional group), but may or may not be derivable from the parent compound. A “derivative” differs from an “analog” or “analogue” in that a parent compound may be the starting material to generate a “derivative,” whereas the parent compound may not necessarily be used as the starting material to generate an “analog.”

In some aspects, the therapeutic agent may be an anti-fibrotic drug. Anti-fibrosis pharmacological mechanisms of action include reduction in local fibroblast proliferation, reduction in local inflammation, and reductions in fibrous tissue growth factors. Anti-fibrotic drugs include, for example, triamcinolone, tranilast, halofuginone, montelukast, zafirlukast, pirfenidone and nintedanib. For example, therapeutic agents such as pirfenidone and nintedanib may slow the progression of scar tissue build up.

Other drugs that may be useful in the present disclosure include, without limitation, glucocorticoids (e.g., cortisol, betamethasone), hirudin, angiopeptin, aspirin, growth factors, antisense agents, anti-cancer agents, anti-proliferative agents, oligonucleotides, and, more generally, anti-platelet agents, anti-coagulant agents, anti-mitotic agents, antioxidants, anti-metabolite agents, anti-chemotactic, and anti-inflammatory agents. Also useful in aspects of the present disclosure are polynucleotides, antisense, RNAi, or siRNA, for example, that inhibit inflammation and/or smooth muscle cell or fibroblast proliferation, contractility, or mobility. Anti-platelet agents can include drugs such as aspirin and dipyridamole. Aspirin is classified as an analgesic, antipyretic, anti-inflammatory and anti-platelet drug. Dipyridamole is a drug similar to aspirin in that it has anti-platelet characteristics. Dipyridamole is also classified as a coronary vasodilator. Anti-coagulant agents for use in aspects of the present disclosure can include drugs such as heparin, protamine, hirudin and tick anticoagulant protein. Anti-oxidant agents can include probucol. Anti-proliferative agents can include drugs such as amlodipine and doxazosin. Anti-mitotic agents and anti-metabolite agents that can be used in aspects of the present disclosure include drugs such as methotrexate, azathioprine, vincristine, adriamycin, and mutamycin. Antibiotic agents for use in aspects of the present disclosure include penicillin, cefoxitin, oxacillin, tobramycin, and gentamicin. Suitable antioxidants for use in aspects of the present disclosure include probucol. Additionally, genes or nucleic acids, or portions thereof can be used as the therapeutic agent in aspects of the present disclosure. Photosensitizing agents for photodynamic or radiation therapy, including various porphyrin compounds such as porfimer, for example, are also useful as drugs in aspects of the present disclosure.

A combination of drugs can also be used in some aspects of the present disclosure. Some of the combinations have additional effects because they have a different mechanism. In aspects, the additional effects may be advantageous for use in the drug coatings described herein. For example, in some aspects, because of the additional effects, the dose of the drug can be reduced. In aspects, combinations of therapeutic agents may reduce complications from using a high dose of the therapeutic agent.

### Coating Layer and Microparticles

In some aspects, the coating layer includes one or more therapeutics therein. In certain aspects, the coating layer may include bioabsorbable polymer microparticles or beads with a therapeutic agent/drug embedded therein, such as a polymer microparticle loaded with therapeutic agent(s) therein.

In some aspects, polymer microparticles may be prepared through forming a polymer solution with a therapeutic suspended therein and causing the polymer to come out of solution, such as through emulsion evaporation. In some aspects, the microparticles are of average size of from 100 nm to 200 μm, including 0.2, 0.3, 0.4, 0.5, 0.6, 0.7, 0.8, 0.9, 1, 1, 2, 3, 4, 5, 6, 7, 8, 9, 10, 20, 30, 40, 50, 60, 70, 80, 90, 110, 120, 130, 140, 150, 160, 170, 180, and 190 μm. In some aspects, the average size may be of about 100 nm to about 300 μm. In some aspects, the average size or D50 may be of about 1 μm to about 300 μm, of about 1 μm to about 100 μm, of about 1 μm to about 50 μm, of about 1 μm to about 40 μm, of about 1 μm to about 30 μm, of about 10 μm to about 300 μm, of about 10 μm to about 100 μm, of about 10 μm to about, of about 10 μm to about 40 μm, or of about 10 μm to about 30 μm. In some aspects, the average size is the D50 value. D50 values can be determined through processes such as laser diffraction. In other aspects of the present disclosure, the polymer microparticles are of a bioabsorbable polymer loaded with a therapeutic, the polymer microparticle being of a uniform size or of a narrow distribution of size, such that 95% of the polymer microparticles are within 20 percent or less of the average selected size. In some aspects, the polymer microparticles may have a uniform or narrow distribution size of from 100 nm to 200 μm (±20%), including 0.2, 0.3, 0.4, 0.5, 0.6, 0.7, 0.8, 0.9, 1, 1, 2, 3, 4, 5, 6, 7, 8, 9, 10, 20, 30, 40, 50, 60, 70, 80, 90, 110, 120, 130, 140, 150, 160, 170, 180, and 190 μm (all±20%). In some aspects, the polymer microparticle is loaded or embedded with a therapeutic such that the therapeutic is of from 5 to 75 % by weight of the polymer microparticle (w/w), including 10, 15, 20, 25, 30, 35, 40, 45, 50, 55, 60, 65, and 70 % by weight of the polymer microparticle. In some aspects, the size of polymer microparticles can be controlled through techniques such as flow and rate in microfluidic channels.

In some aspects, the coating layer on the microneedles and/or exterior surface of the medical device may include at least one excipient.

In some aspects, one or more coating layer(s) on the medical device may include an excipient or excipients. In some aspects, the excipient is applied simultaneously with the one or more coating layer(s). In some aspects, the excipient may underlie the drug coating layer. In other aspects, a drug coating layer may include one or more excipients. In other aspects, an excipient may be applied and/or coated on the drug coating layer. In other aspects, an excipient may underlie a second or top coating layer on the medical device. In addition to the therapeutic agent or combination of therapeutic agents, the drug coating, according to some aspects, may include at least one excipient. In one aspect, the drug coating may include multiple excipients, for example, two, three, or four excipients.

Selection of the excipient or combination thereof may be based on the therapeutic agent microparticle composition and/or coating solvent(s) used. As identified herein, the excipient or combination thereof can be mixed with the therapeutic agent, hydrophobic polymers/small molecules, hydrophilic materials and/or coating solvent(s) to form a coating mixture, which is coated onto the exterior surface of a medical device. Alternatively or additionally, certain aspects may include applying the excipient(s) to the exterior surface of the medical device separately. In some aspects, the excipient or combination thereof may be applied to the medical device before the therapeutic agent dissolved in the coating solvent. In some aspects, the excipient or combination thereof may be applied to the medical device after the therapeutic agent dissolved in the coating solvent. Without being bound by theory, the chosen excipient or combination thereof may be part of a coating mixture that adheres to the medical device such that the coating particles do not fall off during handling and/or interventional procedure. Alternatively or additionally, the chosen excipient or combination thereof, when applied prior to or subsequently after the therapeutic agent, coating solvent, or coating solvents, should adhere to the medical device such that the coating particles do not fall off during handling and/or interventional procedure.

The relative amount of the therapeutic agent and the one or more excipients in the drug coating may vary depending on applicable circumstances. The optimal amount of the one or more excipients can depend upon, for example, the particular therapeutic agent and other excipients selected, the critical micelle concentration of the surface modifier if it forms micelles, the hydrophilic-lipophilic-balance (HLB) of the excipients, the one or more excipients' octanol-water partition coefficient (P), the melting point of the excipients, the water solubility of the excipients and/or therapeutic agent, the surface tension of water solutions of the surface modifier, etc. Other considerations will further inform the choice of specific proportions of the excipients. These considerations include the degree of bioacceptability of the excipients and the desired dosage of therapeutic agent to be provided.

In some aspects, the excipient may include a polymer. The polymer may be an anionic polymer. Examples of anionic polymers include polyglutamic acid or any block polymers containing the same, polyacrylic acid or any block polymers containing the same, polymethylacrylic acid or any block polymers containing same, polystyrene sulfonate or any block polymers containing the same, heparin, hyaluronic acid, and alginate. Without being bound by theory, if the therapeutic agent is cationic in nature, a drug coating including an anionic polymer may allow for the therapeutic agent to be retained for sustained drug release. Similarly, a cationic polymer for an anionic therapeutic agent may allow for the therapeutic agent to be retained for sustained drug release.

In further aspects, the excipient may be a biodurable polymer. As set forth herein, a biodurable polymer may include a polymer that is well-tolerated and/or non-reactive when contacted to a subject or immune-reactive cells thereof and is resistant to erosion and/or enzymatic degradation and/or dissolution within the subject or the circulatory system thereof. Biodurable polymers include polyethylene terephthalate (PET), nylon 6,6, polyurethane (PU), polytetrafluoroethylene (PTFE), polyethylene (PE, low density and high density and ultra-high molecular weight, UHMW), polysiloxanes (silicones) and poly(methylmethacrylate) (PMMA) and Poly(vinylidene fluoride-co-hexafluoropropylene) (PVDF-HFP). In some aspects, the excipient may be PVDF-HFP. Without being bound by theory, utilizing a biodegradable polymers allows for the reduction or elimination of incomplete drug release. In further aspects, the excipient may be a biodegradable polymer. As set forth herein, a biodegradable polymer may include a polymer that is well-tolerated and/or non-reactive when contacted to a subject or immune-reactive cells thereof and is prone to erosion and/or enzymatic degradation and/or dissolution within the subject or the circulatory system thereof over a course of time. Examples of biodegradable polymers include polylactic acid polymers (PLA, PLLA, PDLA, PDLLA), polycaprolactone (PCL), poly lactic-co-glycolic Acid (PLGA), and poly(ethylene glycol) methyl ether-block-poly(lactide-co-glycolide) (PLGA-b-mPEG).

In aspects, the weight ratio of the polymer to the therapeutic agent may be from 5:1 to 8:1, from 5:1 to 7:1, from 5:1 to 6:1, from 6:1 to 8:1, from 6:1 to 7:1, or from 7:1 to 8:1.

Suitable excipients that can be used in some aspects of the present disclosure include, without limitation, organic and inorganic pharmaceutical excipients, natural products and derivatives thereof (such as sugars, vitamins, amino acids, peptides, proteins, and fatty acids), surfactants (anionic, cationic, non-ionic, and ionic), and mixtures thereof. The following list of excipients useful in the present disclosure is provided for exemplary purposes only and is not intended to be comprehensive. Many other excipients may be useful for purposes of the present disclosure, such as polyglutamic acid, polyacrylic acid, hyaluronic acid, alginate, PVA, PVP, Pluronic (PEO-PPO-PEO), cellulose, CMC, HPC, starch, chitosan, human serum albumin (HSA), phospholipids, fatty acid, fatty acid esters, triglycerides, beeswax, cyclodextrin, polysorbates, polyethylene glycol, polyvinylpyrrolidone (PVP) and aliphatic polyesters.

In some aspects, the excipients may feature a drug affinity part. The excipients of the present disclosure may feature a hydrophilic part. As is understood in the art, the terms “hydrophilic” and “hydrophobic” are relative terms. To function as an excipient in some aspects of the present disclosure, the excipient is a compound that includes polar or charged hydrophilic moieties as well as non-polar hydrophobic (lipophilic) moieties. The hydrophilic part can accelerate diffusion and increase permeation of the therapeutic agent into tissue. The hydrophilic part of the excipient may facilitate rapid movement of therapeutic agent off the expandable medical device during deployment at the target site by preventing hydrophobic drug molecules from clumping to each other and to the device, increasing drug solubility in interstitial spaces, and/or accelerating drug passage through polar head groups to the lipid bilayer of cell membranes of target tissues.

Exemplary excipients for application in the present disclosure may include chemical compounds with one or more hydroxyl, amino, carbonyl, carboxyl, acid, amide or ester moieties. Hydrophilic chemical compounds with one or more hydroxyl, amino, carbonyl, carboxyl, acid, amide or ester moieties having a molecular weight less than 5,000 to 10,000 are preferred in certain aspects. In other aspects, molecular weight of the excipient with one or more hydroxyl, amino, carbonyl, carboxyl, acid, amide, or ester moieties is preferably less than 1000 to 5,000, or more preferably less than 750 to 1,000, or most preferably less than 750. In these aspects, the molecular weight of the excipient is less than that of the therapeutic agent to be delivered.

In some aspects, the one or more excipients may be selected from amino alcohols, alcohols, amines, acids, amides and hydroxyl acids in both cyclo-and linear-aliphatic and aromatic groups. Examples include L-ascorbic acid and its salt, D-glucoascorbic acid and its salt, tromethamine, triethanolamine, diethanolamine, meglumine, glucamine, sodium docusate, urea, amine alcohols, glucoheptonic acid, glucomic acid, hydroxyl ketone, hydroxyl lactone, gluconolactone, glucoheptonolactone, glucooctanoic lactone, gulonic acid lactone, mannoic lactone, ribonic acid lactone, lactobionic acid, glucosamine, glutamic acid, benzyl alcohol, benzoic acid, hydroxybenzoic acid, propyl 4-hydroxybenzoate, lysine acetate salt, gentisic acid, lactobionic acid, lactitol, sorbitol, glucitol, sugar phosphates, glucopyranose phosphate, sugar sulphates, sugar alcohols, sinapic acid, vanillic acid, vanillin, methyl paraben, propyl paraben, xylitol, 2-ethoxyethanol, sugars, galactose, glucose, ribose, mannose, xylose, sucrose, lactose, maltose, arabinose, lyxose, fructose, cyclodextrin, (2-hydroxypropyl)-cyclodextrin, acetaminophen, ibuprofen, retinoic acid, lysine acetate, gentisic acid, catechin, catechin gallate, tiletamine, ketamine, propofol, lactic acids, acetic acid, salts of any organic acid and amine described above, polyglycidol, glycerol, multiglycerols, galactitol, di(ethylene glycol), tri(ethylene glycol), tetra(ethylene glycol), penta(ethylene glycol), di(propylene glycol), tri(propylene glycol), tetra(propylene glycol, and penta(propylene glycol), and combinations thereof. Some of the chemical compounds with one or more hydroxyl, amine, carbonyl, carboxyl, amide or ester moieties described herein are very stable under heating, survive an ethylene oxide sterilization process, and/or do not react with the therapeutic agent during sterilization.

In some aspects, the one or more excipients may be selected from amino acids and salts thereof. For example, the excipient may be one or more of alanine, arginine, asparagines, aspartic acid, cysteine, cystine, glutamic acid, glutamine, glycine, histidine, proline, isoleucine, leucine, lysine, methionine, phenylalanine, serine, threonine, tryptophan, tyrosine, valine, and derivatives thereof are. Certain amino acids, in their zwitterionic form and/or in a salt form with a monovalent or multivalent ion, have polar groups, relatively high octanol-water partition coefficients, and are useful in some facets of the present disclosure. In the context of the present disclosure “low-solubility amino acid” refers to amino acid having a solubility in unbuffered water of less than about 4% (40 mg/ml). These include cystine, tyrosine, tryptophan, leucine, isoleucine, phenylalanine, asparagine, aspartic acid, glutamic acid, and methionine.

Amino acid dimers, sugar-conjugates, and other derivatives may also be considered for excipients. Through simple reactions well known in the art hydrophilic molecules may be joined to hydrophobic amino acids, or hydrophobic molecules to hydrophilic amino acids, to make additional excipients useful in aspects of the present disclosure. Catecholamines, such as dopamine, levodopa, carbidopa, and DOPA, are also useful as excipients.

In some aspects, the excipient may be of a material that is at a glass transition temperature at 37° C. or higher. As identified herein, providing a material on the medical device that transitions to a sticky or tacky state in situ within the vessel of the subject allows for adhering the coating to the vessel wall. Such materials may include hydrogenated coconut oil, coconut oil, mineral oil, cetyl alcohol, petrolatum, petroleum jelly, decanol, soft paraffin, tridecanol, dodecanol, long chain saturated fatty acids, long chain unsaturated fatty acids, fatty acid esters, fatty acid ethers, witepsol, solid lipids, methyl stearate, triglycerides, glyceryl monostearate, glyceryl palmitostearate, stearic acid, palmitic acid, decanoic acid, behenic acid, beeswax, carnauba wax, paraffin, fatty acid triglycerides, fatty acid alcohols or combinations thereof.

In some aspects, the excipients may be liquid additives. One or more liquid excipients may be used in the medical device coating to improve the integrity of the coating. Without being bound by theory, a liquid excipient can improve the compatibility of the therapeutic agent in the coating mixture. The liquid excipients used in aspects of the present disclosure is not a solvent. The solvents such as ethanol, methanol, dimethylsulfoxide, and acetone, will be evaporated after the coating is dried. In other words, the solvent will not stay in the coating after the coating is dried. In contrast, the liquid excipients in aspects of the present disclosure will stay in the coating after the coating is dried. The liquid excipient is liquid or semi-liquid at room temperature and one atmosphere pressure. The liquid excipient may form a gel at room temperature. In some aspects, the liquid excipient may be a non-ionic surfactant. Examples of liquid excipients include PEG-fatty acids and esters, PEG-oil transesterification products, polyglyceryl fatty acids and esters, Propylene glycol fatty acid esters, PEG sorbitan fatty acid esters, and PEG alkyl ethers as mentioned above. Some examples of a liquid excipient are Tween 80, Tween 81, Tween 20, Tween 40, Tween 60, Solutol HS 15, Cremophor RH40, and Cremophor EL&ELP.

In some aspects, the excipient may be a surfactant; a chemical compound with one or more hydroxyl, amine, carbonyl, carboxyl, amides or ester moieties; or both. Exemplary surfactants may be chosen from PEG fatty esters, PEG omega-3 fatty esters and alcohols, glycerol fatty esters, sorbitan fatty esters, PEG glyceryl fatty esters, PEG sorbitan fatty esters, sugar fatty esters, PEG sugar esters, Tween 20, Tween 40, Tween 60, p-isononylphenoxypolyglycidol, PEG laurate, PEG oleate, PEG stearate, PEG glyceryl laurate, PEG glyceryl oleate, PEG glyceryl stearate, polyglyceryl laurate, polyglyceryl oleate, polyglyceryl myristate, polyglyceryl palmitate, polyglyceryl-6 laurate, polyglyceryl-6 oleate, polyglyceryl-6 myristate, polyglyceryl-6 palmitate, polyglyceryl-10 laurate, polyglyceryl-10 oleate, polyglyceryl-10 myristate, polyglyceryl-10 palmitate, PEG sorbitan monolaurate, PEG sorbitan monolaurate, PEG sorbitan monooleate, PEG sorbitan stearate, PEG oleyl ether, PEG lauryl ether, Tween 20, Tween 40, Tween 60, Tween 80, octoxynol, monoxynol, tyloxapol, sucrose monopalmitate, sucrose monolaurate, decanoyl-N-methylglucamide, n-decyl-β-D-glucopyranoside, n-decyl-β-D-maltopyranoside, n-dodecyl β-D-glucopyranoside, n-dodecyl-β-D-maltoside, heptanoyl-N-methylglucamide, n-heptyl-β-D-glucopyranoside, n-heptyl-β-D-thioglucoside, n-hexyl-β-D-glucopyranoside, nonanoyl-N-methylglucamide, n-nonyl-β-D-glucopyranoside, octanoyl-N-methylglucamide, n-octyl-β-D-glucopyranoside, octyl-β-D-thioglucopyranoside and their derivatives. In some aspects, the excipients may include one of sodium docusate sorbitol, urea, BHT, BHA, PEG-sorbitan monolaureate, petrolatum, methyl stearate or a combination thereof.

In some aspects, one or more of a surfactant or a small water-soluble molecule (the chemical compounds with one or more hydroxyl, amine, carbonyl, carboxyl, amides or ester moieties) with the therapeutic agent are in certain cases superior to only utilizing the therapeutic agent and a single excipient. By incorporating the one or more additional excipients, the drug coating may have increased stability during transit and rapid drug release when pressed against tissues of the lumen wall at the target site of therapeutic intervention when compared to some formulations comprising the therapeutic agent and only one excipient. Furthermore, the miscibility and compatibility of the therapeutic agent with the excipient or the drug coating with the medical device, generally, is improved by the presence of the one or more additional excipients. For example, a surfactant may allow for improved coating uniformity and integrity.

In some aspects, the coating layer(s) may include multiple excipients, and one excipient is more hydrophilic than one or more of the other excipients. In another embodiment, the coating layer may include multiple excipients, and one excipient has a different structure from that of one or more of the other excipients. In yet another aspect, the coating layer includes multiple excipients. Some aspects of the present disclosure may include a mixture of at least two additional excipients, for example, a combination of one or more surfactants and one or more chemical compound with one or more hydroxyl, amine, carbonyl, carboxyl, amides or ester moieties. For example, therapeutic agents may bind to extremely water-soluble small molecules more poorly than surfactants, which can lead to suboptimal coating uniformity and integrity. Some surfactants may adhere so strongly to the therapeutic agents and the surface of the medical device that the therapeutic agent is not able to rapidly release from the surface of the medical device at the target site. On the other hand, some water-soluble small molecules (with one or more hydroxyl, amine, carbonyl, carboxyl, amides or ester moieties) adhere so poorly to the medical device that they release therapeutic agents before it reaches the target site, for example, into serum during the transit of a coated balloon catheter to the site targeted for intervention. By incorporating a mixture of multiple excipients, the coating layer may have improved properties over a formulation with only one excipient or no excipient.

In some aspects, the one or more additional excipients may include an antioxidant. An antioxidant is a molecule capable of slowing or preventing the oxidation of other molecules. Oxidation reactions can produce free radicals and/or peroxides, which start chain reactions and may cause degradation of therapeutic agents. Antioxidants terminate these chain reactions by removing free radicals and inhibiting oxidation of the active agent by being oxidized themselves. Antioxidants are used as the one or more additional excipients in certain aspects to prevent or slow the oxidation of the therapeutic agents in the coatings for medical devices. Antioxidants are a type of free radical scavengers. The antioxidant may be used alone or in combination with other additional excipients in certain aspects and may prevent degradation of the active therapeutic agent during sterilization or storage prior to use. Some representative examples of antioxidants that may be used in the drug coatings of the present disclosure include, without limitation, oligomeric or polymeric proanthocyanidins, polyphenols, polyphosphates, polyazomethine, high sulfate agar oligomers, chitooligosaccharides obtained by partial chitosan hydrolysis, polyfunctional oligomeric thioethers with sterically hindered phenols, hindered amines such as, without limitation, p-phenylene diamine, trimethyl dihydroquinolones, and alkylated diphenyl amines, substituted phenolic compounds with one or more bulky functional groups (hindered phenols) such as tertiary butyl, arylamines, phosphites, hydroxylamines, and benzofuranones. Also, aromatic amines such as p-phenylenediamine, diphenylamine, and N,N′ disubstituted p-phenylene diamines may be utilized as free radical scavengers. Other examples include, without limitation, butylated hydroxytoluene (“BHT”), butylated hydroxyanisole (“BHA”), L-ascorbate (Vitamin C), Vitamin E, herbal rosemary, sage extracts, glutathione, resveratrol, ethoxyquin, rosmanol, isorosmanol, rosmaridiphenol, propyl gallate, gallic acid, caffeic acid, p-coumeric acid, p-hydroxy benzoic acid, astaxanthin, ferulic acid, dehydrozingerone, chlorogenic acid, ellagic acid, propyl paraben, sinapic acid, daidzin, glycitin, genistin, daidzein, glycitein, genistein, isoflavones, and tertbutylhydroquinone. Examples of some phosphites include di(stearyl)pentaerythritol diphosphite, tris(2,4-di-tert.butyl phenyl)phosphite, dilauryl thiodipropionate and bis(2,4-di-tert.butyl phenyl)pentaerythritol diphosphite. Some examples, without limitation, of hindered phenols include octadecyl-3,5,di-tert.butyl-4-hydroxy cinnamate, tetrakis-methylene-3-(3′,5′-di-tert.buty1-4-hydroxyphenyl)propionate methane 2,5-di-tert-butylhydroquinone, ionol, pyrogallol, retinol, and octadecyl-3-(3,5-di-tert.butyl-4-hydroxyphenyl)propionate. An antioxidant may include glutathione, lipoic acid, melatonin, tocopherols, tocotrienols, thiols, Beta-carotene, retinoic acid, cryptoxanthin, 2,6-di-tert-butylphenol, propyl gallate, catechin, catechin gallate, and quercetin. Preferable antioxidants are butylated hydroxytoluene (BHT) and butylated hydroxyanisole (BHA).

### Polymers

In some aspects, the microneedles are of a biodegradable polyester resin. In some aspects, the biodegradable polyester resins include, but are not limited to: aliphatic polyesters such as composed on a diol and dicarboxylic acid typified by poly(ethylene succinate) and poly(butylene succinate); poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, modified polyethylene glycol, copolymers of these, and mixtures of these. It will also be understood that with respect to micro-and/or nano-particles described herein, these may also be composed of similar materials as they offer the desired degradation within a subject with limited to no toxicity.

In some aspects, the present disclosure concerns polymer components such as polymer microneedles and/or polymer microparticles or beads with a therapeutic agent embedded or dispersed therein. In some aspects, it is advantageous to utilize biocompatible polymers. In some aspects, it is useful to utilize bioabsorbable polymers. In some aspects, a bioabsorbable polymer may include a polymer or linked or cross-linked network of one or more of glycolic acid and lactic acid or L-lactic acid, including polyglycolic acid and poly-L-lactic acid. In some aspects, a bioabsorbable polymer may be a combination of polymers, such as a polymer network of a poly-glycolic acid (PGA) and a poly-L-lactic acid (PLLA). Other bioabsorbable polymers that can be utilized in combination or alone include polycaprolactone (PCL), poly-DL-lactic acid (PDLLA), poly(trimethylene carbonate) (PTMC), poly (ester amine)s (PEA), and poly(para-dioxanone) (PPDO). In some aspects, the bioabsorbable polymer may include, either alone or in combination with other bioabsorbable polymers, a polymer combination of lactic acid and glycolic acid, poly-lactic-co-glycolic acid (PLGA). Those skilled in the art will appreciate that PLGA can be of varying percentages of lactic acid and glycolic acid, wherein the higher the amount of lactide units, the longer the polymer can last in situ before degrading. Additional tunable properties with PLGA concern the molecular weight, with higher weights showing increased mechanical strength. In some aspects, the bioabsorbable polymer may be a polymer of appended units, such as appended with an amine, a carboxylic acid, a polyethylene glycol (PEG), or an amino acid. In some aspects, the bioabsorbable polymer is an appended PLGA.

In some aspects, a therapeutic is dispersed in a polymer are prepared by emulsion evaporation, wherein the therapeutic agent and the polymer are mixed in a solvent such as dichloromethane (DCM) or ethyl acetate (EtOAc) and then formed as the solvent evaporates. Size of the microparticles can be controlled by processes such as microfluidic channel size or membrane emulsification. In some aspects, the microparticles may be prepared with an antioxidant as set forth herein. In some aspects, the microparticles are prepared with butylated hydroxytoluene.

In some aspects, the present disclosure concerns methods of preparing and/or applying the coating layer to an exterior surface of a medical device. Such methods may include selecting a solvent (or a mixture of solvents) wherein the bioabsorbable polymer and/or selected therapeutic agent or therapeutic agents are not soluble or are of low solubility; and then mixing all ingredients and forming a suspension that can then be applied to the medical device and then evaporating the solvent(s) or allowing the solvent to evaporate in the surrounding atmosphere.

As used herein, low solubility refers to a material that cannot easily dissolve in a particular solvent, such as being of 1 g/L or less, such as 0.9 g/L, 0.8 g/L, 0.7 g/L, 0.6 g/L, 0.5 g/L, 0.4 g/L, 0.3 g/L, 0.2 g/L, 0.1 g/L, 0.01 g/L, 0.001 g/L, or less. The solvent may include one or more of water, alcohols, ethers, esters, ketones, aromatic solvents, alkanes and solvents containing halogens (such as fluoride and chloride), methanol, ethanol, iso-propanol, acetone, ethyl acetate, benzene, toluene, chloroform, carbon tetrachloride, hexane, cyclohexane, heptane, octane, pentane, acetonitrile, benzene, iso-butanol, n-butanol, tert-butanol, chlorobenzene, cyclohexanone, cyclopentane, dichloromethane, diethyl ether, dioxane, ethyl ether, ethylene dichloride, xylene and a mixtures thereof.

The methods to apply the drug coating solution to a medical device may include dip coating, metering coating, spray coating, electrostatic spray coating, roller coating, spin coating, ink-jet printing, 3D printing, or combinations thereof. After the solvent has evaporated, the pressure sensitive hydrophobic drug coating is left on the balloon surface. Drug dose density on the medical device can vary from 0.1 to 10 μg/mm², including 0.5 to 5 μg/mm² In some aspects, the present disclosure further concerns packaging and/or sterilizing the medical device before use in a subject.

### Coating Solvents

In some aspects, the present disclosure concerns solvents and the selection thereof for applying the coating(s) as set forth herein to the medical device surface(s). Solvents for preparing of the drug coating, which are referred to herein as “coating solvents,” are used to dissolve the therapeutic agent and the additive. The dissolved therapeutic agent and additive in coating solvent together make up a “coating mixture,” which is coated onto the medical device.

In some aspects, the coating solvent may be any solvent or combination of solvents that are suitable to dissolve the hydrophobic material(s) of the drug coating. In other aspects, the coating solvent may be any solvent or combination of solvents that are suitable to dissolve the selected therapeutic agent. In further aspects, the coating solvent may be any solvent or combination of solvents that are suitable to dissolve the hydrophobic material(s) and the therapeutic agent(s). As identified herein, in some aspects, the therapeutic agent is provided to the surface of the medical device by preparing a mixture or slurry of the therapeutic suspended in a solution of the hydrophobic/hydrophilic material dissolved in solvent. The non-dissolved therapeutic may be of a crystalline form, an amorphous form, or loaded within a microparticle as described herein wherein the microparticle and/or the therapeutic loaded therein does not dissolve in the solvent. Evaporation of the solvent from the surface of the medical device therefore leaves the hydrophobic or hydrophilic layer with the therapeutic suspended therein.

In some aspects, coating solvents may include, as examples, any combination of one or more of the following: water; alkanes such as pentane, cyclopentane, hexane, cyclohexane, heptane, and octane; aromatic solvents such as benzene, toluene, and xylene, alcohols such as methanol, ethanol, 2,2,2-trifluroethanol, propanol, and isopropanol, iso-butanol, n-butanol, tert-butanol, diethylamide, ethylene glycol monoethyl ether, transcutol, and benzyl alcohol; ethers such as dioxane, dimethyl ether, ethyl ether, diethyl ether, di-n-propyl ether, diisopropyl ether, t-butyl methyl ether, petroleum ether, and tetrahydrofuran; esters/acetates such as methyl acetate, ethyl acetate, isobutyl acetate, i-propyl acetate, and n-butyl acetate; ketones such as acetone, acetonitrile, diethyl ketone, cyclohexanone, and methyl ethyl ketones, methyl isobutyl ketone; chlorinated hydrocarbons such as chloroform, dichloromethane, ethylene dichloride; carbon tetrachloride, and chlorobenzene; dioxane; tetrahydrofuran; dimethylformamide; acetonitrile; dimethylsulfoxide; 1,6-dioxane; N, N-Dimethylacetamide (DMA); diethylene glycol; diglyme; 1,2-dimethoxy ethane; hexamethylphosphoramide; and mixtures such as water/ethanol, water/acetone, water/methanol, water/tetrahydrofuran. The amount of coating solvent used depends on the coating process and viscosity, as the amount of solvent may affect the uniformity of the drug coating even though the coating solvent will be evaporated.

In other aspects, two or more solvents, two or more therapeutic agents, two or more polymer microparticle, two or more additives, or, optionally, two or more additional additives may be used in the coating solution or coating mixture. In particular aspects, a hydrophobic polymeric material or a hydrophilic polymeric material may be used as an additive in the coating mixture.

Various techniques may be used for applying a coating solution or coating mixture to a medical device such as metering, casting, spinning, spraying, dipping (immersing), rolling, ink jet printing, 3D printing, electrostatic techniques, plasma etching, vapor deposition, and combinations of these processes. Choosing an application technique principally depends on the viscosity and surface tension of the coating solution or coating mixture. In aspects of the present disclosure, metering, dipping and spraying may be preferred because it makes it easier to control the uniformity of the thickness of the drug coating as well as the concentration of the therapeutic agent applied to the medical device. Regardless of whether the coating solution or coating mixture is applied by spraying or by dipping or by another method or combination of methods, each layer may be applied to the medical device in multiple application steps in order to control the uniformity and the amount of therapeutic substance and additive applied to the medical device.

Each applied layer may have a thickness from 0.1 μm to 15 μm, from 0.1 μm to 10 μm, from 0.1 μm to 5 μm, from 0.1 μm to 1 μm, from 1 μm to 15 μm, from 1 μm to 10 μm, from 1 μm to 5 μm, from 5 μm to 15 μm, from 5 μm to 10 μm, or from 10 μm to 15 μm. The total number of layers applied to the medical device is in a range of from 1 to 50, from 1 to 40, from 1 to 30, from 1 to 20, from 1 to 10, from 10 to 50, from 10 to 40, from 10 to 30, from 10 to 20, from 20 to 50, from 20 to 40, from 20 to 30, from 30 to 40, or from 40 to 50. In some aspects, only one layer is applied to the medical device. In some aspects, more than one layer is applied to the medical device. The total thickness of the coating may be from 0.1 μm to 200 μm, from 0.1 μm to 150 μm, from 0.1 μm to 100 μm, from 0.1 μm to 50 μm, from 0.1 μm to 10 μm, from 0.1 μm to 1 μm, from 1 μm to 200 μm, from 1 μm to 150 μm, from 1 μm to 100 μm, from 1 μm to 50 μm, from 1 μm to 10 μm, from 10 μm to 200 μm, from 10 μm to 150 μm, from 10 μm to 100 μm, from 10 μm to 50 μm, from 50 μm to 200 μm, from 50 μm to 150 μm, from 50 μm to 100 μm, from 100 μm to 200 μm, from 100 μm to 150 μm, or from 150 μm to 200 μm. In other aspects, the secondary water-soluble coat is applied after the drug-coating solvent has evaporated. In further aspects, the secondary water-soluble coating is applied before the solvent of the drug-coating layer has evaporated.

In addition to layers that comprise the coating layer and a secondary water-soluble coating, the medical device may include one or more intermediate layers or top layers. In some aspects, the intermediate or top layer may be advantageous in order to promote adhesion of the drug coating to the medical device, be an additional layer comprising the additive, or prevent premature drug loss during the device delivery process before deployment at the target site.

In one exemplary example, an application device that may be used is a paint jar attached to an air brush, such as a Badger Model 150, supplied with a source of pressurized air through a regulator (Norgren, 0 to 160 psi). When using such an application device, once the brush hose is attached to the source of compressed air downstream of the regulator, the air may be applied. The pressure may be adjusted to approximately 15 psi to 25 psi, and the nozzle condition may be checked by depressing the trigger. Prior to spraying, both ends of a relaxed, expandable medical device may be fastened to the fixture by two resilient retainers, i.e., alligator clips, and the distance between the clips may be adjusted so that the expandable medical device remains in a relaxed condition, for example, a deflated, folded, or an inflated or partially inflated, unfolded condition. The rotor may be then energized and the spin speed adjusted to the desired coating speed, about 40 rpm. With the expandable medical device rotating in a substantially horizontal plane, the spray nozzle may be adjusted so that the distance from the nozzle to the expandable medical device is about 1 inch to 4 inches. First, the coating solution or coating mixture may be sprayed substantially horizontally with the brush being directed along the expandable medical device from the distal end of the expandable medical device to the proximal end and then from the proximal end to the distal end in a sweeping motion at a speed such that one spray cycle occurred in about three expandable medical device rotations. The expandable medical device may be repeatedly sprayed with the coating solution, followed by drying, until an effective amount of the drug is deposited on the expandable medical device. It should be understood that this description of an application device, fixture, and spraying technique is exemplary only. Any other suitable spraying or other technique may be used for coating the expandable medical device, particularly for coating the balloon of a balloon catheter or stent delivery system or stent.

In one additional example of the present disclosure, the expandable medical device may be expanded, such as inflated or partially inflated, and the coating solution or coating mixture may be applied to the expanded expandable medical device, for example by spraying, and then the expandable medical device may be dried and subsequently relaxed or allowed to compress. For example, if the expandable medical device is a balloon, the balloon is dried, deflated, and folded. Drying may be performed under vacuum.

After the medical device is sprayed with the coating solution or coating mixture, the coated medical device may be subjected to a drying in which the coating solvent is evaporated. This produces, on the expandable medical device, a coating matrix containing the therapeutic agent and the additive. One example of a drying technique may include placing the coated expandable medical device into an oven at approximately 20° C. or higher for approximately 24 hours. Another example may include air drying. Any other suitable method of drying the coating solution may be used. The time and temperature may vary with particular additives and therapeutic agents.

In further aspects, the medical device may undergo a sterilization process, such as through exposure to ethylene oxide, steam, dry heat, radiation, vaporized hydrogen peroxide, chlorine dioxide, vaporized peracetic acid, ozone, supercritical carbon dioxide, and/or nitrogen dioxide.

### Drug Eluting Stents

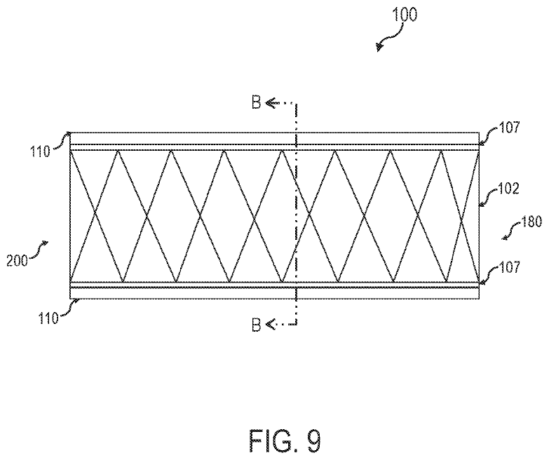

In some aspects, the medical device is drug eluting stent 100. Referring to the example embodiment of FIG. 9, a drug eluting stent 100 has a proximal end 180 and a distal end 200. The drug eluting stent 100 may include any suitable base stent 102 for desired use, including conventional stents known to one of ordinary skill in the art. The base stent 102 may be made of any suitable biocompatible metal alloy. Examples of biocompatible metal alloys may include stainless steel, Nitinol or Elgiloy. In aspects, the shape memory characteristics of Nitinol may allow the base stent 102 to self-expand when placed in a tubular body vessel at normal body temperature.

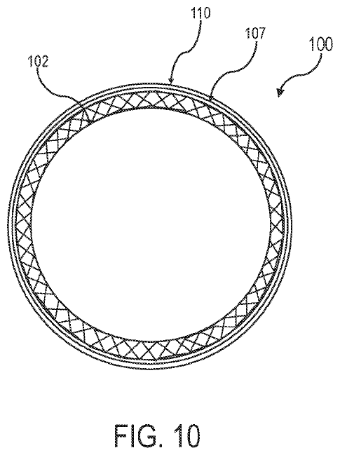

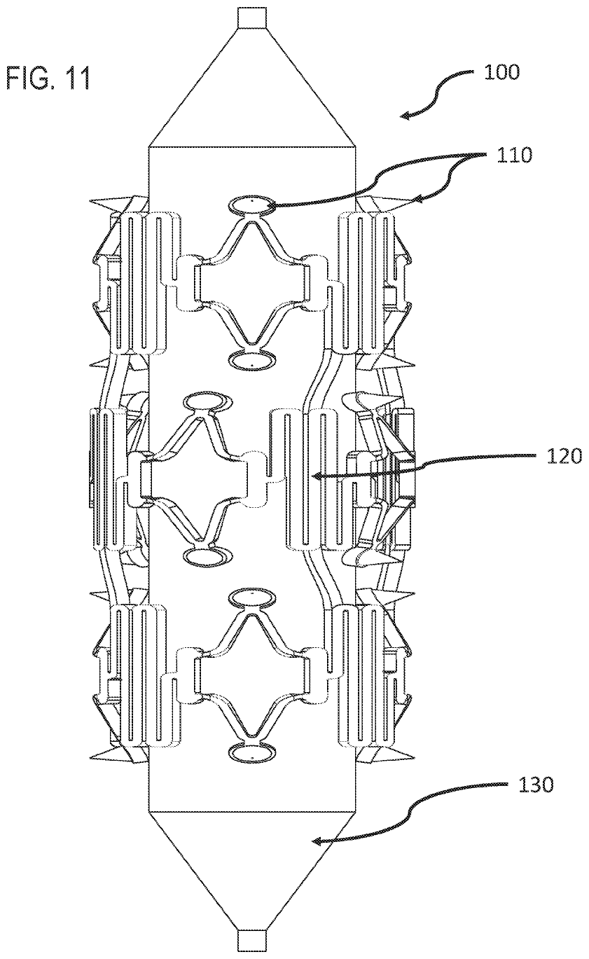

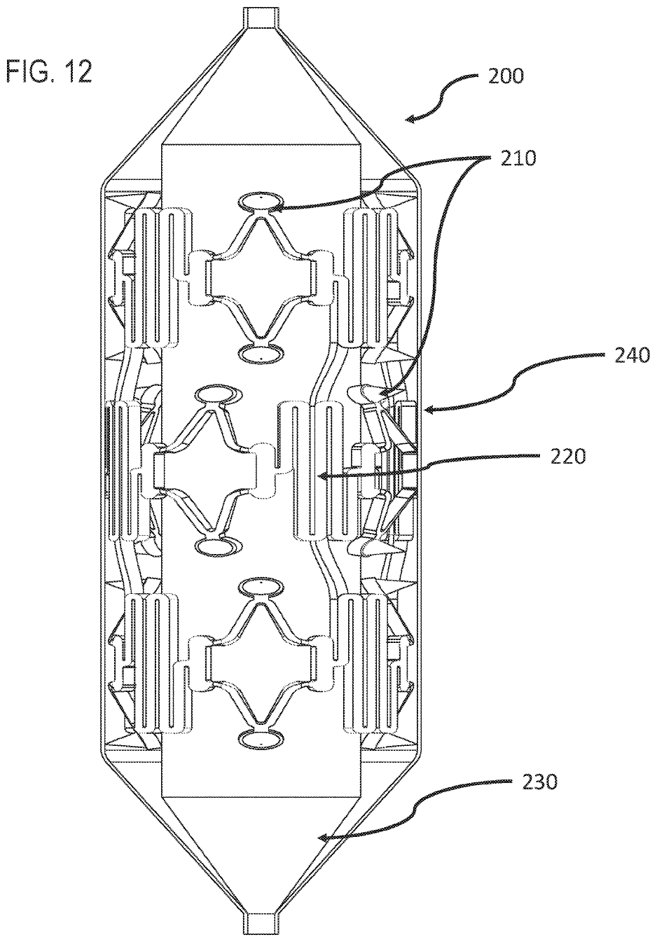

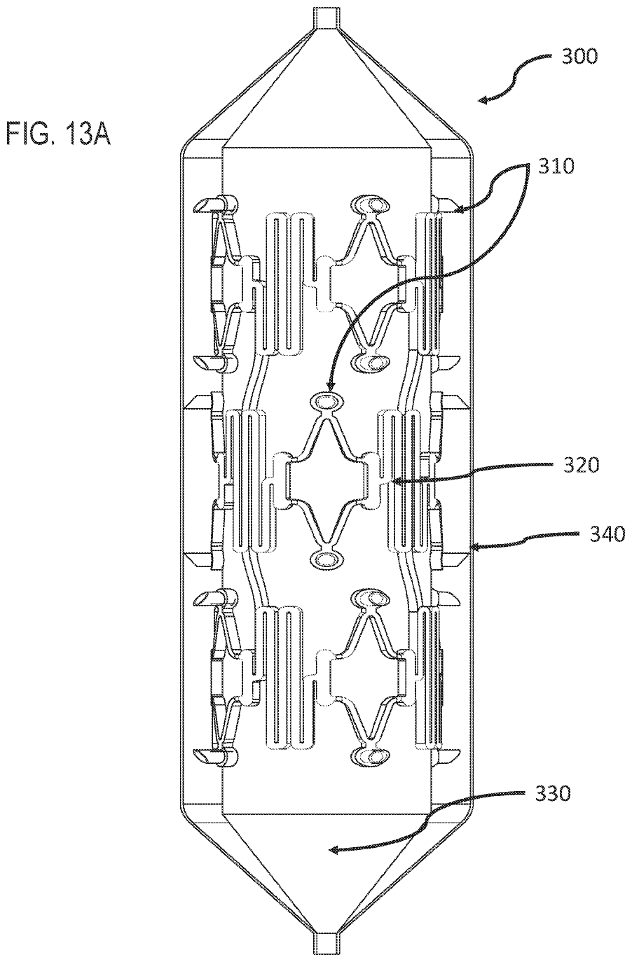

Various aspects of the drug eluting stent 100 of FIG. 9 are illustrated through the cross sections along line B-B of FIG. 9 in FIG. 10. In some example aspects, the cross section B-B of FIG. 3 may be as depicted according to FIG. 10, in which the drug coating layer 110 is applied directly onto an exterior surface 107 of the base stent 102. In some aspects subsequently described, the exterior surface 107 may undergo a surface modification. In aspects where the exterior surface 107 is a modified exterior surface, the exterior surface 107 has been subjected to a surface modification, such as a fluorine plasma treatment, which decreases a surface free energy of the exterior surface 107 before application of the drug coating layer 110. Subjecting the exterior surface to a surface modification may decreases the surface free energy of the exterior surface before application of the coating layer and affect the release kinetics of drug in the coating layer from the balloon, the crystallinity of the drug layer, the surface morphology of the coating and particle shape, or the particle size of drug of a therapeutic layer in the coating layer, drug distribution on the surface.

In aspects in which the cross section B-B of FIG. 9 is as depicted according to FIG. 10, the drug eluting stent 100 includes a drug coating layer 100 applied over an exterior surface 107 of the base stent 102. The drug coating layer 110 itself includes a therapeutic agent and an additive. In one particular embodiment, the drug coating layer 110 comprises a kinase inhibitor, an anti-fibrotic drug therapeutic agent, a polymer, and one or more additional additives. In further aspects, the drug coating layer 110 does not include a polymer.

In other aspects, two or more therapeutic agents are used in combination in the drug coating layer 110. In other aspects, the device may include a top layer (not shown) overlying the drug coating layer 100. In some aspects, a top coat layer may be advantageous in order to prevent premature drug loss during the device delivery process before deployment at the target site.

### EXAMPLES

### Example 1

A polymer solution with a therapeutic agent at a desired concentration can be prepared and then solidified by removing the solvent. The polymer can be formed in a mold or shaped as a solid. In some embodiments, the polymer is cast as the stent body with microneedles attached. In other embodiments, the polymer is provided as needles and then attached to a stent body. In some embodiments, the needles are cast as part of the expandable balloon with fabricated holes in the surrounding stent to allow the microneedle to expand beyond the stent body during expansion.

The stent will then be implanted. For testing purposes, the stent will be implanted in the artery of a porcine model. The stent will then be expanded by expanding a balloon to radially push out the stent. Control stents with no microneedles will be used as well for comparison. The animals will be sacrificed at various time points and the arteries assessed for morphometric cross-section vessel area, lumen area, percentage of stenosis, presence of granulomas, scored for endothelialization, red blood association with struts, scored for inflammation and similar. It is expected that the microneedle stent will perform better.

While particular aspects have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

It is appreciated that all reagents are obtainable by sources known in the art unless otherwise specified.

It is also to be understood that this disclosure is not limited to the specific aspects and methods described herein, as specific components and/or conditions may, of course, vary. Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference is made in detail to exemplary compositions, aspects and methods of the present disclosure, which constitute the best modes of practicing the disclosure presently known to the inventors. The Figures are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular embodiments of the disclosure, but is not meant to be a limitation upon the practice thereof. The following claims, including all equivalents thereof, are intended to define the scope of the disclosure.

1. A medical device comprising a radially-expandable scaffold structure with at least one microneedle positioned circumferentially thereon, wherein the microneedle comprises a coating layer on at least a portion of an outer surface, wherein the coating layer comprises at least one therapeutic agent.

2. The medical device of claim 1, wherein the radially-expandable scaffold structure comprises a stent.

3. The medical device of claim 1, wherein radially-expandable scaffold structure is adhered to an underlying balloon.

4. The medical device of claim 1, wherein the at least one microneedle is attached to the radially-expandable scaffold structure at a hinge or joint structure thereof.

5. The medical device of claim 1, wherein the at least one microneedle is attached to the radially-expandable scaffold structure along an arm thereof.

6. The medical device of claim 1, wherein the at least one microneedle is adhered or welded to the radially-expandable scaffold structure.

7. The medical device of claims 1, wherein the microneedle is part of the radially-expandable scaffold structure.

8. The medical device of claim 7, wherein there is no point of connection between the radially-expandable scaffold structure and the at least one microneedle.

9. The medical device of claim 1, wherein the at least one microneedle extends through the radially-expandable scaffold structure from an external surface of an underlying balloon.

10. The medical device of claim 1, wherein the at least one microneedle is of the same material as the radially-expandable scaffold.

11. The medical device of claim 1, wherein the at least one microneedle is of biopolymer material, wherein the biopolymer material is selected from the group consisting of poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, modified polyethylene glycol, or a copolymer or mixture thereof and wherein optionally the therapeutic agent is embedded therein.

12. The medical device of claim 1, wherein the microneedle is of a metal selected from stainless steel, titanium, nickel, tantalum, platinum, palladium, or nitinol.

13. The medical device of claim 1, wherein the at least one microneedle is connected to an outer surface of the radially-expandable scaffold structure through a detachable junction.

14. The medical device of claim 1, further comprising a hydrophilic-coated thermoplastic layer that covers the coating layer, the radially-expandable scaffold structure, and the at least one microneedle.

15. The medical device of claim 14, wherein the hydrophilic-coated thermoplastic layer comprises a biopolymer material selected from the group consisting of poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, modified polyethylene glycol, or a copolymer or mixture thereof.

16. The medical device of claim 1, wherein the coating layer further comprises nanoparticles with the at least one therapeutic agent loaded therein.

17. The medical device of claim 16, wherein the nanoparticles comprise a biopolymer selected from poly(ethylene succinate), poly(butylene succinate), poly(hydroxyalkanoate), poly(caprolactone), poly(D-and/or L-lactic acid), poly(glycolic acid), poly(3-hydroxybutyric acid), poly(3-hydroxyvaleric acid), poly(3-hydroxycaproic acid), polyvinyl alcohol, dextran sulfate, silk fibroin, albumin, polyethylene oxide, hyaluronic acid, chitosan, polyvinylpyrrolidone, biodegradable elastic polyurethane, modified polyethylene glycol, or a copolymer or mixture thereof.

18. The medical device of claim 1 wherein the therapeutic agent is paclitaxel, rapamycin, daunorubicin, 5-fluorouracil, doxorubicin, bosutinib, ceritinib, crizotinib, gefitinib, ruxolitinib, imatinib, axitinib, nilotinib, trametinib, afatinib, ibrutinib, cabozantinib, imatinib, lenvatinib, sunitinib, regorafenib, sorafenib, vandetanib, dasatinib, pazopanib, Y27632, CA3, verteporfin, VGLL4 peptide, nintedanib, avapritinib, abemaciclib, erdafitinib, fedratinib, palbociclib, pemigatinib, irinotecan, bevasizumab, cetuxamab, biolimus (biolimus A9), everolimus, zotarolimus, tacrolimus, dexamethasone, prednisolone, corticosterone, cisplatin, vinblastine, lidocaine, bupivacaine, xanthine, aminophylline, sildenafil, tadalafil, vardenafil, udenafil, avanafil, dipyridamole, quinazoline, paraxanthine, papaverine, mesembrenone, rolipram, ibudilast, piclamilast, luteolin, drotaverine, roflumilast, apremilast, crisaborole, inamrinone, milrinone, enoximone, anagrelide, cilostazol, pimobendan, erythro-9-(2-hydroxy-3-nonyl)adenine), (2-[(3,4-dimethoxyphenyl)methyl]-7-[(1R)-1-hydroxyethyl]-4-phenylbutyl]-5-methyl-imidazo[5,1-f][1,2,4]triazin-4(1H)-one), oxindole, (9-(6-phenyl-2-oxohex-3-yl)-2-(3,4-dimethoxybenzyl)-purin-6-one), 3-isobutyl-1-methylxanthine, pentoxifylline, theobromine, triamciclone, tranilast, halofuginone, montelukast, zafirlukast, pirfenidone, nintedanib, theophylline, or a combination thereof.

19. The medical device of claim 1 wherein the coating layer further comprises an excipient chosen from a fatty acid, a fatty acid ester, polylactic acid (PLLA, PDLA, PDLLA), polycaprolactone (PCL), sodium docusate, PLGA, PLGA-b-mPEG, polyglutamic acid, polyacrilic acid, hyaluronic acid, alginate, PVA, PVP, Pluronic (PEO-PPO-PEO), cellulose, CMC, HPC, starch, chitosan, human serum albumin (HSA), phospholipids, fatty acid, fatty acid esters, triglycerides, beeswax, cyclodextrin, Tween 20, Tween 80, TPGS, SLS, butylated hydroxytoluene, butylated hydroxyanisole (BHA), vitamin E, vitamin E succinate, and sorbitol esters.

20-82. (canceled)
